(19) United States
(12) Patent Application Publication
(10) Pub. No.: US 2014/0199781 A1
(43) Pub. Date: Jul. 17, 2014

(54) NON-INVASIVE METHODS FOR DIAGNOSING CHRONIC ORGAN TRANSPLANT REJECTION

(71) Applicants: David M. Briscoe, Sharon, MA (US); Kevin P. Daly, Needham, MA (US); Ananth Karumanchi, Chestnut Hill, MA (US); Michael Seifert, Clayton, MO (US)

(72) Inventors: David M. Briscoe, Sharon, MA (US); Kevin P. Daly, Needham, MA (US); Ananth Karumanchi, Chestnut Hill, MA (US); Michael Seifert, Clayton, MO (US)

(73) Assignees: Beth Israel Deaconess Medical Center, Inc., Boston, MA (US); Children's Medical Center Corporation, Boston, MA (US)

(21) Appl. No.: 14/003,313

(22) PCT Filed: Mar. 8, 2012
(86) PCT No.: PCT/US12/28275

§ 371 Date: Feb. 19, 2014

Related U.S. Application Data
(60) Provisional application No. 61/450,604, filed on Mar. 8, 2011.

(51) Int. Cl. G01N 33/536

(52) U.S. Cl. CPC G01N 33/536
USPC 436/501

(57) ABSTRACT

Presented herein are methods of diagnosing or assessing an individual at increased risk of developing chronic rejection, or chronic allograft vasculopathy, based on analysis the individual's biomarker profile.

### RELATED APPLICATION

The present application claims the benefit under 35 U.S.C. §119(e) of U.S. provisional patent application 61/450,604, filed Mar. 8, 2011, the entire content of which is incorporated herein by reference.

### GOVERNMENT FUNDING

This application was made with government support under grant Nos. UL1 RR 025758, 5U01AI063623, 5U01AI063594, 3R01AI046756-10S1, T32 HL07572, and T32DK007726, awarded by National Institutes of Health. The United States government has certain rights in the invention.

### FIELD OF THE INVENTION

The present invention relates to diagnostic and predictive medical technologies, particularly to methods of identifying an individual as having or at risk of developing chronic organ transplant rejection.

### BACKGROUND OF THE INVENTION

Allograft rejection is an inflammatory reaction that can occur rapidly after solid organ transplantation and is associated with a characteristic cellular and humoral attack on the graft. Allograft rejection can also occur chronically, as a result of an insidious immunological process involving delayed type hypersensitivity mechanisms. Both the early acute, and the later chronic rejection processes, are mediated by the recipient's immunological response to donor antigen, which is initiated and coordinated by T cells. All forms of rejection also require the activation of other cell types including B cells and macrophages as well as the induced expression of molecules that enable the trafficking of destructive effector cells to allograft tissue.

Chronic rejection is an indolent but progressive form of primarily immunologic injury to an allograft, which slowly compromises organ function. Although the clinical symptoms of chronic rejection are dependent on the function of the specific organ allograft, the most common histologic manifestation is a progressive narrowing of the arteries, referred to as graft vascular disease. Chronic rejection can begin weeks, months, or years after transplantation, particularly in inadequately immunosuppressed patients with late-onset allograft rejection. In general, the incidence of chronic rejection increases with time after transplantation and eventually affects a majority of solid organ allografts, except for liver allografts. By five years after transplantation, chronic rejection affects up to 80% of lung allograft recipients, 30%-40% of heart, kidney, and pancreas allograft recipients, and about 5% of liver recipients (Demetris, A. J. et al. Pathophysiology of Chronic Allograft Rejection, see website: medscape.org/viewarticle/418503_{—}2, incorporated herein by reference).

A diagnosis of chronic rejection ultimately relies on an invasive biopsy of the transplanted organ after the disease has progressed. In addition, cardiac allograft vasculopathy is seen with chronic cardiac allograft rejection and can be diagnosed by selective coronary angiography or CT angiography. For example, following heart transplantation, individuals undergo either coronary angiography or intravascular ultrasound, both of which are expensive, highly invasive, and limited in their ability to detect early onset disease. The prediction of chronic rejection may enable the development of, as well as the use of, therapeutics to halt its progression. Currently, there is no way to predict if an organ transplant recipient will develop chronic rejection, and there is no non-invasive way to diagnose a patient with chronic rejection. Development of such predictive and diagnostic tests may be particularly useful in pediatric heart transplant recipients, wherein invasive techniques are associated with higher adverse event rates and are technically limited by patient size.

### SUMMARY OF THE INVENTION

In one aspect, the present disclosure provides a method of identifying an organ transplant recipient individual as having or at increased risk of developing chronic organ transplant rejection, comprising: (i) determining a level(s) of vascular endothelial growth factor-C(VEGF-C), vascular endothelial growth factor-A (VEGF-A), platelet factor-4 (PF4), Artemin, Urokinase Plasminogen Activator (uPA), Vasohibin, or Angiopoietin-2, or any combination thereof in a sample from the individual; (ii) comparing the level(s) of VEGF-C, VEGF-A, PF4, Artemin, uPA, Vasohibin, or Angiopoietin-2, or any of the combinations thereof in the sample to a predetermined value(s) for VEGF-C, VEGF-A, PF4, Artemin, uPA, Vasohibin, or Angiopoietin-2, or any of the combinations thereof, respectively; and (iii) identifying the individual as having or at increased risk of developing chronic organ transplant rejection if the level(s) of VEGF-C, VEGF-A, PF4, Artemin, uPA, Vasohibin, or Angiopoietin-2, or any of the combinations thereof in the sample is above (greater than) the predetermined value(s) for VEGF-C, VEGF-A, PF4, Artemin, uPA, Vasohibin, or Angiopoietin-2, or any of the combinations thereof, respectively. In some embodiments, the method comprises assaying for the level(s) of VEGF-C, VEGF-A, PF4, Artemin, uPA, Vasohibin, or Angiopoietin-2, or any of the combinations thereof in the sample.

In some embodiments, a method of identifying an organ transplant recipient individual as having or at increased risk of developing chronic organ transplant rejection comprises: (i) determining a level(s) of vascular endothelial growth factor-C (VEGF-C), vascular endothelial growth factor-A (VEGF-A), or platelet factor-4 (PF4), or any combination thereof in a sample from the individual; (ii) comparing the level(s) of VEGF-C, VEGF-A, or PF4, or any of the combinations thereof in the sample to a predetermined value(s) for VEGF-C, VEGF-A, or PF4, or any of the combinations thereof, respectively; and (iii) identifying the individual as having or at increased risk of developing chronic organ transplant rejection if the level(s) of VEGF-C, VEGF-A, or PF4, or any of the combinations thereof in the sample is above the predetermined value(s) for VEGF-C, VEGF-A, or PF4, or any of the combinations thereof, respectively. In some embodiments, the method comprises assaying for the level(s) of VEGF-C, VEGF-A, or PF4, or any of the combinations thereof in the sample.

In some embodiments, a method of identifying an organ transplant recipient individual as having or at increased risk of developing chronic organ transplant rejection comprises: (i) determining a level of VEGF-C and a level of VEGF-A in a sample from the individual; (ii) comparing the level of VEGF-C and the level of VEGF-A in the sample to a predetermined value for VEGF-C and a predetermined value for VEGF-A, respectively; and (iii) identifying the individual as having or at increased risk of developing chronic organ transplant rejection if the level of VEGF-C and the level of VEGF-A in the sample is above the predetermined value for VEGF-C and above the predetermined value for VEGF-A, respectively.

In some embodiments, a method of identifying an organ transplant recipient individual as having or at increased risk of developing chronic organ transplant rejection comprises: (i) determining a level of VEGF-C and a level of PF4 in a sample from the individual; (ii) comparing the level of VEGF-C and the level of PF4 in the sample to a predetermined value for VEGF-C and a predetermined value for PF4, respectively; and (iii) identifying the individual as having or at increased risk of developing chronic organ transplant rejection if the level of VEGF-C and the level of PF4 in the sample is above the predetermined value for VEGF-C and above the predetermined value for PF4, respectively.

In some embodiments, a method of identifying an organ transplant recipient individual as having or at increased risk of developing chronic organ transplant rejection comprises: (i) determining a level of VEGF-A and PF4 in a sample from the individual; (ii) comparing the level of VEGF-A and the level of PF4 in the sample to a predetermined value for VEGF-A and a predetermined value for PF4, respectively; and (iii) identifying the individual as having or at increased risk of developing chronic organ transplant rejection if the level of VEGF-A and the level of PF4 in the sample is above the predetermined value for VEGF-A and above the predetermined value for PF4, respectively.

In some embodiments, a method of identifying an organ transplant recipient individual as having or at increased risk of developing chronic organ transplant rejection comprises: (i) determining a level of VEGF-C, a level of VEGF A, and a level of PF4 in a sample from the individual; (ii) comparing the level of VEGF-C, the level of VEGF-A, and the level of PF4 in the sample to a predetermined value for VEGF-C, a predetermined value for VEGF-A, and a predetermined value for PF4 respectively; and (iii) identifying the individual as having or at increased risk of developing chronic organ transplant rejection if the level of VEGF-C, the level of VEGF-A, and the level of PF4 in the sample is above the predetermined value for VEGF-C, above the predetermined value for VEGF-A, and above the predetermined value for PF4, respectively.

In another aspect, the present disclosure provides a method of identifying an individual as having or at increased risk of developing chronic organ transplant rejection, comprising: (i) comparing the level(s) of VEGF-C, VEGF-A, PF4, Artemin, uPA, Vasohibin, or Angiopoietin-2, or any combination thereof in a sample from an organ transplant recipient individual to a predetermined value(s) for VEGF-C, VEGF-A, PF4, Artemin, uPA, Vasohibin, or Angiopoietin-2, or any of the combinations thereof, respectively; and (ii) identifying the individual as having or at increased risk of developing chronic organ transplant rejection if the level(s) of VEGF-C, VEGF-A, PF4, Artemin, uPA, Vasohibin, or Angiopoietin-2, or any of the combinations thereof in the sample is above the predetermined value(s) for VEGF-C, VEGF-A, PF4, Artemin, uPA, Vasohibin, or Angiopoietin-2, or any of the combinations thereof, respectively.

In some embodiments, In some embodiments, a method of identifying an organ transplant recipient individual as having or at increased risk of developing chronic organ transplant rejection comprises: (i) comparing the level(s) of VEGF-C, VEGF-A, or PF4, or any combination thereof in a sample from an organ transplant recipient individual to a predetermined value(s) for VEGF-C, VEGF-A, or PF4, or any of the combinations thereof, respectively; and (ii) identifying the individual as having or at increased risk of developing chronic organ transplant rejection if the level(s) of VEGF-C, VEGF-A, or PF4, or any of the combinations thereof in the sample is above the predetermined value(s) for VEGF-C, VEGF-A, or PF4, or any of the combinations thereof, respectively.

In some embodiments, a method of identifying an organ transplant recipient individual as having or at increased risk of developing chronic organ transplant rejection comprises: (i) comparing a level of VEGF-C and a level of VEGF-A in a sample from the individual to a predetermined value for VEGF-C and a predetermined value for VEGF-A, respectively; and (ii) identifying the individual as having or at increased risk of developing chronic organ transplant rejection if the level of VEGF-C and the level of VEGF-A in the sample is above the predetermined value for VEGF-C and above the predetermined value for VEGF-A, respectively.

In some embodiments, a method of identifying an organ transplant recipient individual as having or at increased risk of developing chronic organ transplant rejection comprises: (i) comparing a level of VEGF-C and a level of PF4 in a sample from the individual to a predetermined value for VEGF-C and a predetermined value for PF4, respectively; and (ii) identifying the individual as having or at increased risk of developing chronic organ transplant rejection if the level of VEGF-C and the level of PF4 in the sample is above the predetermined value for VEGF-C and above the predetermined value for PF4, respectively.

In some embodiments, a method of identifying an organ transplant recipient individual as having or at increased risk of developing chronic organ transplant rejection comprises: (i) comparing a level of VEGF-A and a level of PF4 in a sample from the individual to a predetermined value for VEGF-A and a predetermined value for PF4, respectively; and (ii) identifying the individual as having or at increased risk of developing chronic organ transplant rejection if the level of VEGF-A and the level of PF4 in the sample is above the predetermined value for VEGF-A and above the predetermined value for PF4, respectively.

In some embodiments, a method of identifying an organ transplant recipient individual as having or at increased risk of developing chronic organ transplant rejection comprises: (i) comparing a level of VEGF-C, a level of VEGF-A, and a level of PF4 in a sample from the individual to a predetermined value for VEGF-C, a predetermined value VEGF-A, and a predetermined value PF4 respectively; and (ii) identifying the individual as having or at increased risk of developing chronic organ transplant rejection if the level of VEGF-C, the level of VEGF-A, and the level of PF4 in the sample is above the predetermined value for VEGF-C, above the predetermined value for VEGF-A, and above the predetermined value for PF4, respectively.

In any one of the aspects and embodiments described herein, the predetermined value(s) for VEGF-C, VEGF-A, PF4, Artemin, uPA, Vasohibin, or Angiopoietin-2, or any combination thereof may be from a control population. In some aspects and embodiments, the level(s) of VEGF-C, VEGF-A, PF4, Artemin, uPA, Vasohibin, or Angiopoietin-2, or any combination thereof is determined using R&D Systems Proteome Profiler™ Human Angiogenesis Array Kit (ARY007) (Minneapolis, Minn.). In some aspects and embodiments, the predetermined value for VEGF-C is a serum densitometric unit of about 270. In some aspects and embodiments, the predetermined value for VEGF-A is a serum densitometric unit of about 2200.

In yet another aspect, the present disclosure provides a method of identifying an individual as having or at increased risk of developing chronic organ transplant rejection, comprising: (i) determining a level of VEGF-C in a sample from the individual; (ii) comparing the level of VEGF-C in the sample to a predetermined value for VEGF-C; and (iii) identifying the individual as having or at increased risk of developing chronic organ transplant rejection if the level of VEGF-C in the sample is above the predetermined value for VEGF-C; or determining a level of a biomarker other than VEGF-C if the level of VEGF-C in the sample is at or below the predetermined for VEGF-C to identify if the individual has or is at increased risk of developing chronic organ transplant failure. In some embodiments, the biomarker other than VEGF-C is VEGF-A. In some embodiments, the method comprises assaying for the level(s) of VEGF-C and/or VEGF-A in the sample.

In some embodiments, the method further comprises: (i) comparing a level of VEGF-A in the sample to a predetermined value for VEGF-A; (ii) comparing a level of Angiostatin in the sample to a predetermined value for Angiostatin if the level of VEGF-A in the sample is above the predetermined value for VEGF-A, or comparing a level of Angiopoietin-1 in the sample to a predetermined value for Angiopoietin-1 if the level of VEGF-A in the sample is at or below the predetermined value for VEGF-A; and (iii) identifying the individual as having or at increased risk of developing chronic organ transplant rejection if the level of Angiostatin in the sample is at or below the predetermined value for Angiostatin or if the level of Angiopoietin-1 in the sample is at or below the predetermined value for Angiopoietin-1. In some embodiments, the method comprises determining the level(s) of VEGF-A, Angiostatin, or Angiopoietin-1, or any combination thereof in the sample from the individual. In some embodiments, the method comprises assaying for the level(s) of VEGF-A, Angiostatin, or Angiopoietin-1, or any combination thereof in the sample from the individual.

In still another aspect, the present disclosure provides a method of identifying an individual as having or at increased risk of developing chronic organ transplant rejection, comprising: (i) comparing the level of VEGF-C in a sample from the individual to a predetermined value for VEGF-C; and (ii) identifying the individual as having or at increased risk of developing chronic organ transplant rejection if the level of VEGF-C in the sample is above the predetermined value for VEGF-C; or determining a level of a biomarker other than VEGF-C if the level of VEGF-C in the sample is at or below the predetermined for VEGF-C to identify if the individual has or is at increased risk of developing chronic organ transplant failure. In some embodiments, the biomarker other than VEGF-C is VEGF-A.

In some embodiments, the method further comprises: (i) comparing a level of VEGF-A in the sample to a predetermined value for VEGF-A; (ii) comparing a level of Angiostatin in the sample to a predetermined value for Angiostatin if the level of VEGF-A in the sample is above the predetermined value for VEGF-A, or comparing a level of Angiopoietin-1 in the sample to a predetermined value for Angiopoietin-1 if the level of VEGF-A in the sample is at or below the predetermined value for VEGF-A; and (iii) identifying the individual as having or at increased risk of developing chronic organ transplant rejection if the level of Angiostatin in the sample is at or below the predetermined value for Angiostatin or if the level of Angiopoietin-1 in the sample is at or below the predetermined value for Angiopoietin-1. In some embodiments, the method comprises determining the level(s) of VEGF-A, Angiostatin, or Angiopoietin-1, or any combination thereof in the sample from the individual. In some embodiments, the method comprises assaying for the level(s) of VEGF-A, Angiostatin, or Angiopoietin-1, or any combination thereof in the sample from the individual.

In yet a further aspect, the present disclosure provides a method of identifying an organ transplant recipient individual as having or at increased risk of developing chronic organ transplant rejection, comprising: (i) determining a level of VEGF-A in a sample from the individual; (ii) comparing the level of VEGF-A in the sample to a predetermined value for VEGF-A; (iii) comparing a level of Angiostatin in the sample to a predetermined value for Angiostatin if the level of VEGF-A in the sample is above the predetermined value for VEGF-A, or comparing a level of Angiopoietin-1 in the sample to a predetermined value for Angiopoietin-1 if the level of VEGF-A in the sample is at or below the predetermined value for VEGF-A; and (iv) identifying the individual as having or at increased risk of developing chronic organ transplant rejection if the level of Angiostatin in the sample is at or below the predetermined value for Angiostatin or if the level of Angiopoietin-1 in the sample is at or below the predetermined value for Angiopoietin-1. In some embodiments, the method comprises determining the level(s) of Angiostatin, Angiopoietin-1, or both in the sample from the individual. In some embodiments, the method comprises assaying for the level(s) of VEGF-A, Angiostatin, or Angiopoietin-1, or any combination thereof in the sample from the individual.

In another aspect, the present disclosure provides a method of identifying an organ transplant recipient individual as having or at increased risk of developing chronic organ transplant rejection, comprising: (i) comparing a level of VEGF-A in a sample from the individual to a predetermined value for VEGF-A; (ii) comparing a level of Angiostatin in the sample to a predetermined value for Angiostatin if the level of VEGF-A in the sample is above the predetermined value for VEGF-A, or comparing a level of Angiopoietin-1 in the sample to a predetermined value for Angiopoietin-1 if the level of VEGF-A in the sample is at or below the predetermined value for VEGF-A; and (iii) identifying the individual as having or at increased risk of developing chronic organ transplant rejection if the level of Angiostatin in the sample is at or below the predetermined value for Angiostatin or if the level of Angiopoietin-1 in the sample is at or below the predetermined value for Angiopoietin-1. In some embodiments, the method comprises determining the level(s) of VEGF-A, Angiostatin, or Angiopoietin-1, or any combination thereof in the sample from the individual. In some embodiments, the method comprises assaying for the level(s) of VEGF-A, Angiostatin, or Angiopoietin-1, or any combination thereof in the sample from the individual.

In any one of the aspects and embodiments described herein, the predetermined value for VEGF-C, VEGF-A, Angiostatin, or Angiopoietin-1, of any combination thereof may be a value or corresponds to a value from a control population.

In any one of the aspects and embodiments described herein, the predetermined value for VEGF-C, VEGF-A, Angiostatin, or Angiopoietin-1, or any combination thereof may be determined using R&D Systems Proteome Profiler™ Human Angiogenesis Array Kit (ARY007) (Minneapolis, Minn.). In some aspects and embodiments, the predetermined value for VEGF-C is a serum densitometric unit of about 270. In some aspects and embodiments, the predetermined value for VEGF-A is a serum densitometric unit of about 2200. In some aspects and embodiments, the predetermined value for Angiostatin is a serum densitometric unit of about 23000. In some aspects and embodiments, the predetermined value for Angiopoietin-1 is a serum densitometric unit of about 470.

In any one of the aspects and embodiments described herein, the sample may be a tissue, tissue aspirate, or bodily fluid. In some aspects and embodiments, the sample is blood, plasma, or serum. In some aspects and embodiments, the sample is urine. In some aspects and embodiments, the sample is saliva.

In any one of the aspects and embodiments described herein, the level(s) (biomarker level) may be a protein level or a nucleic acid level (e.g., expression level). In some aspects and embodiments, the nucleic acid level is a DNA level or a RNA level.

In any one of the aspects and embodiments described herein, the organ may be heart, heart valve, kidney, liver, lung, bone, bone marrow, ligament, tendon, intestine, cornea, skin, or bladder. In some aspects and embodiments, the individual is a heart transplant recipient.

In one aspect, the present disclosure provides a kit, comprising: (i) means for measuring a protein level(s) or a nucleic acid level(s) of VEGF-C, VEGF-A, PF4, Artemin, uPA, Vasohibin, or Angiopoietin-2, or any combination thereof; (ii) a predetermined value(s) for VEGF-C, VEGF-A, PF4, Artemin, uPA, Vasohibin, or Angiopoietin-2, or any of the combinations thereof; and (iii) instructions for comparing the protein level(s) or the nucleic acid level(s) of VEGF-C, VEGF-A, PF4, Artemin, uPA, Vasohibin, or Angiopoietin-2, or any of the combinations thereof to the predetermined value(s) for VEGF-C, VEGF-A, PF4, Artemin, uPA, Vasohibin, or Angiopoietin-2, or any of the combinations thereof, wherein (i)-(iii) are packaged into one unit.

In another aspect, the present disclosure provides a kit, comprising: (i) means for measuring a protein level(s) or a nucleic acid level(s) of VEGF-C, VEGF-A, Angiostatin, or Angiopoietin-1, or any combination thereof; (ii) a predetermined value(s) for VEGF-C, VEGF-A, Angiostatin, or Angiopoietin-1, or any of the combinations thereof; and (iii) instructions for comparing the protein level(s) or the nucleic acid level(s) of VEGF-C, VEGF-A, Angiostatin, or Angiopoietin-1, or any of the combinations thereof to the predetermined value(s) for VEGF-C, VEGF-A, or PF4, or any of the combinations thereof, wherein (i)-(iii) are packaged into one unit.

In some aspects and embodiments, the kits described herein further comprise a means for collecting a sample. In aspects and some embodiments, the kits described herein are used for identification of an organ transplant recipient individual having or at increased risk of developing chronic organ transplant rejection. In any one of the kits described herein, the sample may be tissue, tissue aspirate, bodily fluid, blood, plasma, serum, urine, or saliva.

### BRIEF DESCRIPTION OF THE DRAWINGS

FIG. 1 shows several graphs of protein measurements for proteins involved in vascular injury and repair responses associated with cardiac allograft vasculopathy (CAV). Data is displayed using box and whisker plots on a logarithmic scale. Measurements which were below the limit of detection were assigned a value of 1 Densitometric Unit for the purpose of display.

FIG. 2 shows a receiver-operating characteristic (ROC) curve analysis for VEGF-A, VEGF-C, and PF4 (area under the curve (AUC)=0.982, 95% CI: 0.940-1.000).

FIG. 3 depicts a classification and regression tree (CART) used to identify biomarkers for classifying individuals with chronic rejection/chronic allograft vasculopathy; predetermined value (PDV).

### DETAILED DESCRIPTION OF THE INVENTION

The instant disclosure relates in part to biomarker profiles and diagnostic or predictive tests (e.g., non-invasive tests) for identifying organ transplant recipient individuals as having or at increased (greater) risk of developing chronic rejection, a complication of organ transplantation that frequently limits the survival or the organ allograft. Chronic rejection may describe a long-term loss of function in transplanted allografts (i.e., sourced from a genetically non-identical member of the same species as the recipient individual). An allograft may be a tissue, cell, or an organ. The allograft rejection may be due to chronic inflammatory and immune response against the transplanted tissue, cell, or organ. If chronic rejection is associated with fibrosis of the internal blood vessels of the transplanted tissue, cell, or organ, it may be referred to as chronic allograft vasculopathy (CAV). Herein, chronic rejection and CAV may be used interchangeably. In some instances, chronic rejection and/or CAV may also be referred to as chronic organ transplant rejection. It is to be understood that an organ transplant may include an allograft as well as a xenograft (i.e., sourced from another species such as porcine or bovine). Transplantable organs and tissues include, for example, heart, heart valve, kidney, liver, lung, bone, bone marrow, ligament, tendon, intestine, cornea, skin, and bladder. In one embodiment, chronic rejection of a heart is referred to as cardiac allograft vasculopathy.

The instant disclosure is based in part on the discovery that serum levels of a biomarkers vascular endothelial growth factor-C(VEGF-C), vascular endothelial growth factor-A (VEGF-A), platelet factor 4 (PF4), Artemin, Urokinase Plasminogen Activator (uPA), Vasohibin, or Angiopoietin-2, or any combination thereof can be used to diagnose chronic rejection. For example, VEGF-C, VEGF-A, and PF4 can be used to diagnose chronic rejection with about 100% sensitivity and about 94% specificity. The platform used involves the quantification of endothelial injury and repair processes as biomarkers of chronic rejection.

The instant disclosure is also based in part on the discovery that serum levels of a panel of biomarkers, VEGF-C, VEGF-A, Angiostatin, and Angiopoietin-1, or VEGF-A, Angiostatin, and Angiopoietin-1 can be used to diagnose chronic rejection with about 100% sensitivity and about 100% specificity.

In some embodiments, the biomarkers are used to diagnose a transplant organ recipient with chronic rejection. In some embodiments, the biomarkers are used to predict if a candidate organ transplant recipient is at increased (greater) risk or not at increased risk of developing chronic rejection.

Vascular endothelial growth factor-C(VEGF-C) as used in some embodiments herein is a member of the platelet-derived growth factor/vascular endothelial growth factor (PDGF/VEGF) family, is active in angiogenesis, lymphangiogenesis (Karkkauinen, et al. Oncogene (2000) 19(49):5598-5605), and endothelial cell growth and survival, and can also affect the permeability of blood vessels. VEGF-C protein is secreted and undergoes a complex proteolytic maturation, generating multiple processed forms that bind and activate VEGFR-3 receptors. Only the fully processed form can bind and activate VEGFR-2 receptors. The structure and function of this protein is similar to those of vascular endothelial growth factor D (VEGF-D). In some embodiments, VEGF-C may refer to VEGF-C full-length protein, or fragments, antigens, or epitopes thereof, including different isoforms and preproteins of VEGF-C. In other embodiments, VEGF-C may refer to full-length or variant nucleic acid, or nucleic acid truncation. An example of full-length VEGF-C mRNA includes the mRNA defined by National Center for Biotechnology Information (NCBI) accession NM_{—}005429, incorporated herein by reference. An example of full-length VEGF-C protein includes the protein defined by NCBI accession NM_{—}005429, incorporated herein by reference. Also included herein are VEGF-C preproteins, for example, proteins defined by NCBI accession no. NP_{—}005420.1, incorporated herein by reference.

Vascular endothelial growth factor-A (VEGF-A) as used in some embodiments herein is a member of the PDGF/VEGF growth factor family. VEGF-A protein is often found as a disulfide linked homodimer. This protein is a glycosylated mitogen that specifically acts on endothelial cells and has various effects, including mediating increased vascular permeability, inducing angiogenesis, vasculogenesis and endothelial cell growth, promoting cell migration, and inhibiting apoptosis. Elevated levels of this protein is linked to POEMS syndrome, also known as Crow-Fukase syndrome. Mutations in this gene have been associated with proliferative and nonproliferative diabetic retinopathy. Alternatively spliced transcript variants, encoding either freely secreted or cell-associated isoforms, have been characterized, and are included herein. There is also evidence for the use of non-AUG (CUG) translation initiation sites upstream of, and in-frame with the first AUG, leading to additional isoforms, which are included herein. In some embodiments described herein, VEGF-A may refer to VEGF-A full-length protein, or fragments, antigens, or epitopes thereof, including different isoforms and preproteins of VEGF-A. In other embodiments, VEGF-A may refer to full-length or variant nucleic acid, or nucleic acid truncation. An example of full-length VEGF-A mRNA for use herein includes the mRNA defined by NCBI accession NM_{—}001025366, incorporated herein by reference. An example of full-length VEGF-A protein for use herein includes the protein defined by NCBI accession NM_{—}001025366, incorporated herein by reference. Other examples of VEGF-A species for use herein include those defined by NCBI accession nos. NM_{—}001025366.2, NP_{—}001020537.2, NM_{—}001025367.2, NP_{—}001020538.2, NM_{—}001025368.2, NP_{—}001020539.2, NM_{—}001025369.2, NP_{—}001020540.2, NM_{—}001025370.2, NP_{—}001020541.2, NM_{—}001033756.2, NP_{—}001028928.1, NM_{—}001171622.1, NP_{—}001165093.1, NM_{—}001171623.1, NP_{—}001165094.1, NM_{—}001171624.1, NP_{—}001165095.1, NM_{—}001171625.1, NP_{—}001165096.1, NM_{—}001171626.1, NP_{—}001165097.1, NM_{—}001171627.1, NP_{—}001165098.1, NM_{—}001171628.1, NP_{—}001165099.1, NM_{—}001171629.1, NP_{—}001165100.1, NM_{—}001171630.1, NP_{—}001165101.1, NM_{—}001204384.1, NP_{—}001191313.1, NM_{—}001204385.1, NP_{—}001191314.1, NM_{—}003376.5, and NP_{—}003367.4, incorporated herein by reference.

Platelet factor 4 (PF4) as used in some embodiments herein is a small cytokine belonging to the CXC chemokine family that is also known as chemokine (C-X-C motif) ligand 4 (CXCL4). This chemokine is released from alpha-granules of activated platelets during platelet aggregation, and promotes blood coagulation by moderating the effects of heparin-like molecules. It is chemotactic for neutrophils, fibroblasts and monocytes, and interacts with a splice variant of the chemokine receptor CXCR3, known as CXCR3B. The gene for human PF4 is located on human chromosome 4. In some embodiments, PF4 may refer to PF4 full-length protein, or fragments, antigens, or epitopes thereof, including different isoforms and pre-proteins of PF4. In other embodiments, PF4 may refer to full-length or variant nucleic acid, or nucleic acid truncation. An example of full-length PF4 mRNA includes the mRNA defined by NCBI accession NM_{—}002619.2, incorporated herein by reference. An example of full-length PF4 protein includes the protein defined by NCBI accession NP_{—}002610.1, incorporated herein by reference.

Angiostatin as used in some embodiments herein is a naturally occurring protein found in several animal species, including humans. It is an endogenous angiogenesis inhibitor (i.e., it blocks the growth of new blood vessels). Angiostatin is a fragment of a larger protein, plasminogen. In some embodiments plasminogen may replace Angiostatin in any one of the methods described herein. Antibodies used in the methods described herein may recognize and bind to both Angiostatin and plasminogen. Example of full-length plasminogen mRNA and a full-length plasminogen protein as used in some embodiments herein are described by O'Reilly et al. Cell (1994) 79(2):315-28, incorporated herein by reference. Angiostatin is a 38 kDa protein with its N-terminus at AA 98 and its C-terminus at amino acid 440 of plasminogen (O'Reilly et al. (1994)). Other examples of plasminogen and Angiostatin as used in some embodiments herein include NCBI accession nos. NM_{—}000301.3, NP_{—}000292.1, NM_{—}001168338.1, and NP_{—}001161810.1, incorporated herein by reference.

Angiopoietin-1 as used in some embodiments herein is a type of angiopoietin and is encoded by the gene ANGPT1. Angiopoietins are proteins with roles in vascular development and angiogenesis. All angiopoietins bind with similar affinity to an endothelial cell-specific tyrosine-protein kinase receptor. The protein encoded by this gene is a secreted glycoprotein that activates the receptor by inducing its tyrosine phosphorylation. It plays a role in mediating reciprocal interactions between the endothelium and surrounding matrix and mesenchyme. The protein also contributes to blood vessel maturation and stability. There are several Angiopoietin-1 transcript variants, any of which may be used as described herein. An example of a full-length Angiopoietin-1 mRNA variant as used in some embodiments herein includes the mRNA defined by NCBI accession NM_{—}001199859, incorporated herein by reference. An example of full-length Angiopoietin-1 protein variant as used in some embodiments herein includes the protein defined by NCBI accession NM_{—}001199859, incorporated herein by reference. Other Angiopoietin-1 variants as used in some embodiments herein include those defined by NCBI accession nos. NM_{—}001146.3 and NP_{—}001137.2, incorporated herein by reference.

Artemin as used in some embodiments herein is a neurotrophin in the glial cell line-derived neurotophic factor family of ligands which are a group of ligands within the TGF-beta superfamily of signaling molecules. Artemin has been shown in culture to support the survival of a number of peripheral neuron populations and at least one population of dopaminergic central nervous system (CNS) neurons. Its role in the peripheral nervous system (PNS) and CNS is further substantiated by its expression pattern in the proximity of these neurons. This protein is a ligand for the RET receptor and uses GFR-alpha 3 as a coreceptor. In some embodiments, Artemin may refer to Artemin full-length protein, or fragments, antigens, or epitopes thereof, including different isoforms and preproteins of Artemin. In other embodiments, Artemin may refer to full-length or variant nucleic acid, or nucleic acid truncation. An example of full-length Artemin mRNA as used in some embodiments herein includes the mRNA (cDNA clone MGC:71178 IMAGE:5453642) defined by NCBI accession BC062375, incorporated herein by reference. Examples of variant Artemin mRNA as used in some embodiments herein include mRNA defined by NCBI accession NM_{—}001136215 and NM_{—}057091, incorporated herein by reference. Examples of Artemin protein as used in some embodiments herein include proteins defined by NCBI accession AAD13110 and AAD13109, incorporated herein by reference.

Urokinase Plasminogen Activator (uPA or urokinase) as used in some embodiments herein is a serine protease present in, for example, urine, blood, and extracellular matrix. The primary physiological substrate is plasminogen, which is an inactive zymogen form of the serine protease plasmin. Activation of plasmin triggers a proteolysis cascade that, depending on the physiological environment, participates in thrombolysis or extracellular matrix degradation. Urokinase is a 411-residue protein, consisting of three domains: the serine protease domain, the kringle domain, and the growth factor domain. Urokinase is synthesized as a zymogen form (prourokinase or single-chain urokinase), and is activated by proteolytic cleavage between L158 and 1159. The two resulting chains are kept together by a disulfide bond. The most important inhibitors of uPA are the serpins plasminogen activator inhibitor-1 (PAI-1) and plasminogen activator inhibitor-2 (PAI-2), which inhibit the protease activity irreversibly. In the extracellular matrix, urokinase is tethered to the cell membrane by its interaction to the urokinase receptor. In some embodiments, uPA may refer to uPA full-length protein, or fragments, antigens, or epitopes thereof, including different isoforms and preproteins of uPA. In other embodiments, uPA may refer to full-length or variant nucleic acid, or nucleic acid truncation. Examples of nucleotide sequences of uPA as used in some embodiments herein are described by S. J. Degenet al. (J Biol Chem. (1986) 25; 261(15): 6972-85) and W. Thomas et al. (Hum Mol Genet. (1992) 1(2):138). Examples of uPA protein include proteins defined by NCBI accession AAB59510 and AAA98809.

Vasohibin as used in some embodiments herein is an extracellular (EC)-derived protein that operates in a direct negative feedback loop to limit VEGF and fibroblast growth factor (FGF) basic-induced angiogenesis. Vasohibin blocks VEGF or FGF basic-induced Human Umbilical Vein Endothelial Cell (HUVEC) migration and microtubule formation. Vasohibin is anti-angiogenic in the chorioallantoic membrane and corneal micropocket assays. Vasohibin-transfected tumor cells show decreased tumorigenicity, and the resulting tumors have less well developed vasculature than nontransfected cell tumors. Vasohibin also blocks neovascularization in pathological conditions such as retinal ischemia and adventitial angiogenesis following vascular injury. In some embodiments, Vasohibin may refer to Vasohibin full-length protein, or fragments, antigens, or epitopes thereof, including different isoforms and preproteins of Vasohibin. In other embodiments, Vasohibin may refer to full-length or variant nucleic acid, or nucleic acid truncation. An example of a full-length Vasohibin mRNA variant includes the mRNA defined by NCBI accession BC086688, incorporated herein by reference.

The Angiopoietins are protein growth factors that promote angiogenesis, the formation of blood vessels from pre-existing blood vessels. There are now four identified Angiopoietins: Angiopoietin-1, Angiopoietin-2, Angiopoietin-3, and Angiopoietin-4. In addition, there are a number of proteins that are closely related to Angiopoietins (ANGPTL2, ANGPTL3, ANGPTL4, ANGPTL5, ANGPTL6, ANGPTL7). Angiopoietin-1 and Angiopoietin-2 are required for the formation of mature blood vessels. In some embodiments, Angiopoietin-2 may refer to Angiopoietin-2 full-length protein, or fragments, antigens, or epitopes thereof, including different isoforms and preproteins of Angiopoietin-2. In other embodiments, Angiopoietin-2 may refer to full-length or variant nucleic acid, or nucleic acid truncation. An example of a full-length Angiopoietin-2 mRNA includes the mRNA defined by NCBI accession BC143902. An example of a full-length Angiopoietin-2 protein includes the protein defined by NCBI accession AAI43903.1.

In some embodiments, a diagnosis of an organ transplant recipient individual with chronic rejection is made based on a level of VEGF-C in a sample from the individual. The level of VEGF-C may be compared to a predetermined value for VEGF-C. The predetermined value for VEGF-C may be from a matched control population.

In some embodiments, a diagnosis of an organ transplant recipient individual at increased risk of developing chronic rejection is made based on a level of VEGF-C in a sample from the individual. The level of VEGF-C may be compared to a predetermined value for VEGF-C. The predetermined value for VEGF-C may be from a matched control population.

In some embodiments, a diagnosis of an organ transplant recipient individual with chronic rejection is made based on a level of VEGF-A in a sample from the individual. The level of VEGF-A may be compared to a predetermined value for VEGF-A. The predetermined value for VEGF-A may be from a matched control population.

In some embodiments, a diagnosis of an organ transplant recipient individual at increased risk of developing chronic rejection is made based on a level of VEGF-A in a sample from the individual. The level of VEGF-A may be compared to a predetermined value for VEGF-A. The predetermined value for VEGF-A may be from a matched control population.

In some embodiments, a diagnosis of an organ transplant recipient individual with chronic rejection is made based on a level of PF4 in a sample from the individual. The level of PF4 may be compared to a predetermined value for PF4. The predetermined value for PF4 may be from a matched control population.

In some embodiments, a diagnosis of an organ transplant recipient individual at increased risk of developing chronic rejection is made based on a level of PF4 in a sample from the individual. The level of PF4 may be compared to a predetermined value for PF4. The predetermined value for PF4 may be from a matched control population.

In some embodiments, a diagnosis of an organ transplant recipient individual with chronic rejection is made based on a level of Artemin in a sample from the individual. The level of Artemin may be compared to a predetermined value for Artemin. The predetermined value for Artemin may be from a matched control population.

In some embodiments, a diagnosis of an organ transplant recipient individual at increased risk of developing chronic rejection is made based on a level of Artemin in a sample from the individual. The level of Artemin may be compared to a predetermined value for Artemin. The predetermined value for Artemin may be from a matched control population.

In some embodiments, a diagnosis of an organ transplant recipient individual with chronic rejection is made based on a level of uPA in a sample from the individual. The level of uPA may be compared to a predetermined value for uPA. The predetermined value for uPA in may be from a matched control population.

In some embodiments, a diagnosis of an organ transplant recipient individual at increased risk of developing chronic rejection is made based on a level of uPA in a sample from the individual. The level of uPA may be compared to a predetermined value for uPA. The predetermined value for uPA may be from a matched control population.

In some embodiments, a diagnosis of an organ transplant recipient individual with chronic rejection is made based on a level of Vasohibin in a sample from the individual. The level of Vasohibin may be compared to a predetermined value for Vasohibin. The predetermined value for Vasohibin may be from a matched control population.

In some embodiments, a diagnosis of an organ transplant recipient individual at increased risk of developing chronic rejection is made based on a level of Vasohibin in a sample from the individual. The level of Vasohibin may be compared to a predetermined value for Vasohibin. The predetermined value for Vasohibin may be from a matched control population.

In some embodiments, a diagnosis of an organ transplant recipient individual with chronic rejection is made based on a level of Angiopoietin-2 in a sample from the individual. The level of Angiopoietin-2 may be compared to a predetermined value for Angiopoietin-2. The predetermined value for Angiopoietin-2 may be from a matched control population.

In some embodiments, a diagnosis of an organ transplant recipient individual at increased risk of developing chronic rejection is made based on a level of Angiopoietin-2 in a sample from the individual. The level of Angiopoietin-2 may be compared to a predetermined value for Angiopoietin-2. The predetermined value for Angiopoietin-2 may be from a matched control population.

In some embodiments, if the level(s) of VEGF-C, VEGF-A, PF4, Artemin, uPA, Vasohibin, or Angiopoietin-2, or any combination thereof in a sample from an organ transplant recipient individual is above (greater than) a predetermined value(s) for VEGF-C, VEGF-A, PF4, Artemin, uPA, Vasohibin, or Angiopoietin-2, or any of the combinations thereof, respectively, the individual is identified as having or at increased risk of developing chronic rejection, relative to an individual whose level(s) of VEGF-C, VEGF-A, PF4, Artemin, uPA, Vasohibin, or Angiopoietin-2, or any of the combinations thereof is at or below (less than) the predetermined value(s) for VEGF-C, VEGF-A, PF4, Artemin, uPA, Vasohibin, or Angiopoietin-2, or any of the combinations thereof, respectively. If the individual's level(s) of VEGF-C, VEGF-A, PF4, Artemin, uPA, Vasohibin, or Angiopoietin-2, or any of the combinations thereof is at or below the predetermined value(s) for VEGF-C, VEGF-A, PF4, Artemin, uPA, Vasohibin, or Angiopoietin-2, or any of the combinations thereof, respectively, the individual may be identified as not having or not at an increased risk of developing chronic rejection, relative to an individual whose level(s) of VEGF-C, VEGF-A, PF4, Artemin, uPA, Vasohibin, or Angiopoietin-2, or any of the combinations thereof is above the predetermined value(s) for VEGF-C, VEGF-A, PF4, Artemin, uPA, Vasohibin, or Angiopoietin-2, or any of the combinations thereof, respectively. As used herein, a value “above” a predetermined value is a value “greater than” the predetermined value, and a value “at or below” a predetermined value is a value “equal to or less than” the predetermined value.

In some embodiments, if the level of VEGF-C and the level of VEGF-A in a sample from an organ transplant recipient individual is above a predetermined value for VEGF-C and above a predetermined value for VEGF-A, respectively, the individual is identified as having or at increased risk of developing chronic rejection, relative to an individual whose level of VEGF-C and level of VEGF-A is at or below the predetermined value for VEGF-C and at or below the predetermined value for VEGF-A, respectively.

In some embodiments, if the level of VEGF-C and the level of PF4 in a sample from an individual is above a predetermined value for VEGF-C and above a predetermined value for PF4, respectively, the individual is identified as having or at increased risk of developing chronic rejection, relative to an individual whose level of VEGF-C and level of PF4 is at or below the predetermined value for VEGF-C and at or below the predetermined value for PF4, respectively.

In some embodiments, if the level of VEGF-A and the level of PF4 in a sample from an organ transplant recipient individual is above a predetermined value for VEGF-A and above a predetermined value for PF4, respectively, the individual is identified as having or at increased risk of developing chronic rejection, relative to an individual whose level of VEGF-A and level of PF4 is at or below the predetermined value for VEGF-A and at or below the predetermined value for PF4, respectively.

In some embodiments, if the level of VEGF-C in a sample from an organ transplant recipient individual is above a predetermined value for VEGF-C, the individual is identified as having or at increased risk of developing chronic rejection, relative to an individual whose level of VEGF-C is at or below the predetermined value for VEGF-C. If the individual's level of VEGF-C is at or below the predetermined value for VEGF-C, the sample may be used to determine a level of a biomarker other than VEGF-C to identify if the individual has or is at increased risk of developing chronic organ transplant failure.

In some embodiments, identifying an organ transplant recipient individual as having or at increased risk of developing chronic rejection may require further comparison of a level of VEGF-A in the sample from the individual to a predetermined value for VEGF-A, different from the predetermined value used in the comparison of the level of VEGF-C. If the level of VEGF-C is at or below the predetermined value for VEGF-C, then a level of VEGF-A in a sample from the individual is compared to a predetermined value for VEGF-A.

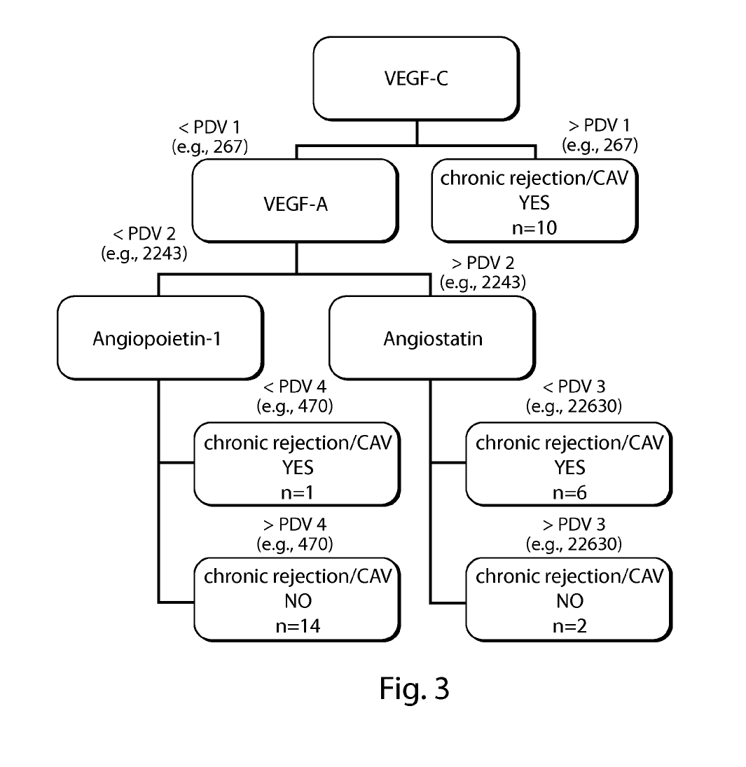

If an analysis of the level of VEGF-A is required, a comparison of the level of Angiostatin and the level of Angiopoietin-1 to a predetermined value for Angiostatin and a predetermined value Angiopoietin-1, respectively, is required. If the level of VEGF-A is above the predetermined value for VEGF-A, then the level of Angiostatin is compared to a predetermined value for Angiostatin. If the level of Angiostatin is at or below the predetermined value for Angiostatin, then the individual is identified as having or at increased risk of developing chronic rejection, relative to an individual whose level of VEGF-A is above the predetermined value for VEGF-A and whose level of Angiostatin is above the predetermined value for Angiostatin. If the level of VEGF-A is at or below the predetermined value for VEGF-A, then the level of Angiopoietin-1 is compared to a predetermined value for Angiopoietin-1. If the level of Angiopoietin-1 is at or below the predetermined value for Angiopoietin-1, then the individual is identified as having or at increased risk of developing chronic rejection, relative to an individual whose level of VEGF-A is at or below the predetermined value for VEGF-A and whose level of Angiopoietin-1 is above the predetermined value for Angiopoietin-1 (FIG. 3, CART diagram).

In yet another embodiment, a level of VEGF-A is compared to a predetermined value for VEGF-A in the absence of (without) a level of VEGF-C comparison. When comparing a level of VEGF-A in a sample from an organ transplant recipient individual to a predetermined value for VEGF-A, if the level of VEGF-A is above the predetermined value for VEGF-A, then the level of Angiostatin is compared to a predetermined value for Angiostatin. If the level of Angiostatin is at or below the predetermined value for Angiostatin, then the individual is identified as having or at increased risk of developing chronic rejection, relative to an individual whose level of VEGF-A is above the predetermined value for VEGF-A and whose level of Angiostatin is above the predetermined value for Angiostatin. If the level of VEGF-A is at or below the predetermined value for VEGF-A, then the level of Angiopoietin-1 is compared to a predetermined value for Angiopoietin-1. If the level of Angiopoietin-1 is at or below the predetermined value for Angiopoietin-1, then the individual is identified as having or at increased risk of developing chronic rejection, relative to an individual whose level of VEGF-A is at or below the predetermined value for VEGF-A and whose level of Angiopoietin-1 is above the predetermined value for Angiopoietin-1.

VEGF-C, VEGF-A, Angiostatin, and Angiopoietin-1 biomarker level(s) may be measures of protein level(s) or nucleic acid level(s). In some embodiments, protein level(s) or nucleic acid level(s) may refer to an expressed level(s). Nucleic acid level(s) may include a DNA level or a RNA level. A protein level or nucleic acid level herein can be from any biological fluid, cell, tissue, organ or portion thereof, from which protein or nucleic acid can be isolated. The protein or nucleic acid can be derived from a biological source by isolation techniques or amplification techniques or a combination of these techniques. A sample can be in a variety of ways known in the art. Samples may be according to standard techniques from all types of biological sources that are usual sources of protein and/or nucleic acid including, but not limited to cells or cellular components which contain protein and/or nucleic acid, biopsies, bodily fluids such as tissue aspirate, blood (e.g., platelets, red and white blood cells), plasma, serum, sputum, stool, urine, cerebrospinal fluid, ejaculate, and all possible combinations thereof. Furthermore, nucleic acids can be amplified or copied such that the sequence information is converted to another nucleic acid form such as RNA, cDNA, cRNA or the like.

“A sample” or “the sample,” as used herein refers to one or more samples from a single individual. For example, levels of VEGF-C in a first serum sample from an individual may be measured, and simultaneously or subsequently, levels of VEGF-A from the same serum sample or from a second serum sample from the same individual may be measured. Similarly, levels of VEGF-C in a first plasma sample from an individual may be measured, and simultaneously or subsequently, levels of VEGF-A from the same plasma sample or from a second plasma sample from the same individual may be measured.

Biomarker levels may be obtained by any art recognized method, and such methods are not limited to those described herein. Typically, the level is determined by measuring the level of VEGF-C, VEGF-A, PF4, Artemin, uPA, Vasohibin, Angiopoietin-2, Angiostatin, and/or Angiopoietin-1 in a body fluid, for example, blood, serum, plasma, lymph, saliva, urine, and the like. The level can be determined by ELISA, or other immunoassays or other conventional techniques for determining the presence of VEGF-C, VEGF-A, PF4, Artemin, uPA, Vasohibin, Angiopoietin-2, Angiostatin, and/or Angiopoietin-1. Conventional methods may include sending a sample(s) of an individual's body fluid to a commercial laboratory for measurement. Methods for measuring the biomarkers are described herein. In one embodiment, concentrations of biomarker proteins can determined using MILLIPLEX® Mag beads on the LUMINEX® XMAP® platform (Millipore). In other embodiments, concentrations of biomarker proteins can be determined using any one or more fluorescent bead assay, or the like, or with any other comparable analyzer. Serum and plasma can be spun down, divided into aliquots, and stored at −80 degrees Celsius to prevent repeated freeze/thaw cycles. Magnetic beads with conjugated capture antibodies specific for the analytes of interest can be mixed together. Working standards and quality controls can be prepared in parallel. Fifty microliters of standards, quality controls, or unknown samples can be incubated with the antibody coated capture beads for several hours or overnight. Each sample can be run in duplicate. A primary and secondary reporter antibody can be incubated with the beads. The assay can be performed as outlined with the MILLIPLEX® Mag kit, or as outlined in any other assay or kit used for a similar purpose. A LUMINEX® analyzer, or other comparable analyzer, can used to identify beads and measure levels of reporter antibody. Analysis can be performed by generating a 5-parameter logarithmic standard curve for each analyte and determining concentrations in the unknown sample based on those curves.

The invention involves comparing the level of a biomarker (e.g., VEGF-C, VEGF-A, PF4, Artemin, uPA, Vasohibin, Angiopoietin-2, Angiostatin, and/or Angiopoietin-1) in a sample from the organ transplant recipient individual to a predetermined value for the particular biomarker. The predetermined value can take a variety of forms. For example, it can be a control level from a control population.

The disclosure involves identifying (e.g., diagnosing) organ transplant recipient individuals as having chronic organ transplant rejection based on a level(s) of a biomarker(s) (e.g., VEGF-C, VEGF-A, PF4, Artemin, uPA, Vasohibin, Angiopoietin-2, or any combination thereof, or VEGF-C, VEGF-A, Angiostatin, and Angiopoietin-1, or VEGF-A, Angiostatin, and Angiopoietin-1) in a sample from the organ transplant recipient individual. The disclosure also involves identifying organ transplant recipient individuals as being at increased risk of developing chronic organ transplant rejection, i.e., identifying organ transplant recipient individuals who do not have chronic organ transplant rejection (e.g., have no signs, symptoms of chronic organ transplant rejection or, for example, have no clinical, histologic, or imaging evidence of chronic organ transplant rejection) but who are at increased/higher risk of developing chronic organ transplant rejection as compared to a control group based on the level(s) of a biomarker(s) (e.g., VEGF-C, VEGF-A, PF4, Artemin, uPA, Vasohibin, Angiopoietin-2, or any combination thereof, or VEGF-C, VEGF-A, Angiostatin, and Angiopoietin-1, or VEGF-A, Angiostatin, and Angiopoietin-1) in a biological sample from the organ transplant recipient individual.

The disclosure involves comparing the level of a biomarker (e.g., VEGF-C, VEGF-A, PF4, Artemin, uPA, Vasohibin, Angiopoietin-2, or any combination thereof, or VEGF-C, VEGF-A, Angiostatin, and Angiopoietin-1, or VEGF-A, Angiostatin, and Angiopoietin-1) in a sample from the organ transplant recipient individual to a predetermined value for the particular biomarker(s). The predetermined value can take a variety of forms. For example, it can be a level (control level) from a control population. A control population can be a matched population, which includes individuals who are similar to the organ transplant recipient individual with respect to, for example, age, weight, sex, race, health or disease condition, demographic characteristics, immunosuppression, and/or genetic background, etc. but who are not diagnosed with chronic organ transplant rejection (e.g., are without signs and/or symptoms of chronic organ transplant rejection). Thus, for example, a control population can be a group of individuals who are age-matched (i.e., same or similar in age) and sex-matched (i.e., same sex).

In some embodiments, the control population comprises a group of individuals who have not undergone organ transplantation (individuals who have not received an organ transplant). In some embodiments, the control population comprises a group of individuals who have undergone organ transplantation but do not have chronic organ transplant rejection. Thus, for example, a control population can be an age-matched, sex-matched group who have not undergone organ transplantation. In some embodiments, the control population comprises an age-matched, sex-matched group of individuals who are organ transplant recipient individuals (have undergone organ transplantation) but who do not have chronic organ transplant rejection. Chronic organ transplant rejection can be diagnosed, for example, by examination of histologic changes in a biopsy(ies) from the transplanted organ. Histologic manifestations or changes associated with or characteristic of chronic organ transplant rejection are known to those of ordinary skill in the art. For example, one histologic manifestation of chronic organ transplant rejection is a progressive narrowing of the arteries, referred to as graft vascular disease.

The predetermined value can be a baseline level from the organ transplant recipient individual before the individual received the organ transplant or shortly after the individual received the transplant (e.g., 1 day, 2 days, 3 days, 4 days, 5 days, 6 days, 1 week, 2 weeks, 3 weeks, 1 month, 2 months, 3 months, 4 months, or more after the individual received the organ transplant). The increase above the predetermined value may be an increase (e.g., statistically significant increase) above the organ transplant recipient individual's baseline level or a fold change (e.g., increase) from the organ transplant recipient individual's baseline level.

The predetermined value can be higher or lower depending on the population from which it is obtained (e.g., collected, taken). For example, a predetermined value for a biomarker from a control population of middle-aged females may be higher than the predetermined value for the same biomarker from younger-aged males. The predetermined value can be a single cut-off value, such as a median or mean. It can be established based upon comparative groups, such as, for example, where the risk in one defined group is double the risk in another defined group. It can be a range, for example, where the tested population is divided equally (or unequally) into groups, such as a low-risk group, a medium-risk group and a high-risk group, or into quartiles, the lowest quartile being individuals with the highest risk and the highest quartile being individuals with the lowest risk, or into tertiles the lowest tertile being individuals with the highest risk and the highest tertile being individuals with the lowest risk. The predetermined value may be a cut-off value which is predetermined by the fact that a group having a VEGF-C, VEGF-A, PF4, Artemin, uPA, Vasohibin, Angiopoietin-2, Angiostatin, and/or Angiopoietin-1, level less than or greater than (depending on the particular biomarker) the cut-off value demonstrates a statistically significant increase risk of developing chronic rejection as compared to a comparative group.

As discussed above, the predetermined value can depend upon the particular population of individuals selected. Accordingly, the predetermined values selected may take into account the category in which an individual falls. Appropriate ranges and categories can be selected with no more than routine experimentation by those of ordinary skill in the art. In some embodiments, the preferred body fluids are serum, plasma, and blood.

In some embodiments, the predetermined value of VEGF-C is about 100 pg/ml of serum. In yet other embodiments, the predetermined value of VEGF-C is about 110 pg/ml of serum, about 120 pg/ml, about 130 pg/ml, about 140 pg/ml, about 150 pg/ml, about 160 pg/ml, about 170 pg/ml, about 180 pg/ml, about 190 pg/ml, about 200 pg/ml, about 210 pg/ml of serum, about 220 pg/ml, about 230 pg/ml, about 240 pg/ml, about 250 pg/ml, about 260 pg/ml, about 270 pg/ml, about 280 pg/ml, about 290 pg/ml, or about 300 pg/ml of serum. In other embodiments, the predetermined value of VEGF-C is about 310 pg/ml of serum, about 320 pg/ml, about 330 pg/ml, about 340 pg/ml, about 350 pg/ml, about 360 pg/ml, about 370 pg/ml, about 380 pg/ml, about 390 pg/ml, or about 400 pg/ml, about 410 pg/ml of serum, about 420 pg/ml, about 430 pg/ml, about 440 pg/ml, about 450 pg/ml, about 460 pg/ml, about 470 pg/ml, about 480 pg/ml, about 490 pg/ml, or about 500 pg/ml of serum. In still other embodiments, the predetermined value of VEGF-C is about 510 pg/ml of serum, about 520 pg/ml, about 530 pg/ml, about 540 pg/ml, about 550 pg/ml, about 560 pg/ml, about 570 pg/ml, about 580 pg/ml, about 590 pg/ml, about 600 pg/ml, about 610 pg/ml of serum, about 620 pg/ml, about 630 pg/ml, about 640 pg/ml, about 650 pg/ml, about 660 pg/ml, about 670 pg/ml, about 680 pg/ml, about 690 pg/ml, or about 700 pg/ml of serum. In further embodiments, the predetermined value of VEGF-C is about 710 pg/ml of serum, about 720 pg/ml, about 730 pg/ml, about 740 pg/ml, about 750 pg/ml, about 760 pg/ml, about 770 pg/ml, about 780 pg/ml, about 790 pg/ml, about 800 pg/ml, about 810 pg/ml of serum, about 820 pg/ml, about 830 pg/ml, about 840 pg/ml, about 850 pg/ml, about 860 pg/ml, about 870 pg/ml, about 880 pg/ml, about 890 pg/ml, or about 900 pg/ml of serum. In other embodiments, the predetermined value of VEGF-C is about 910 pg/ml of serum, about 920 pg/ml, about 930 pg/ml, about 940 pg/ml, about 950 pg/ml, about 960 pg/ml, about 970 pg/ml, about 980 pg/ml, about 990 pg/ml, about 1,000 pg/ml of serum. In yet other embodiments, the predetermined value of VEGF-C is about 2,000 pg/ml, about 3,000 pg/ml, about 4,000 pg/ml, about 5,000 pg/ml, about 6,000 pg/ml, about 7,000 pg/ml, about 8,000 pg/ml, about 9,000 pg/ml, or about 10,000 pg/ml of serum.

In some embodiments, the predetermined value of VEGF-A is about 20 pg/ml of serum. In other embodiments, the predetermined value of VEGF-A is about 30 pg/ml, about 40 pg/ml, about 50 pg/ml, about 60 pg/ml, about 70 pg/ml, about 80 pg/ml, or about 90 pg/ml. In yet other embodiments, the predetermined value of VEGF-A is about 100 pg/ml, about 110 pg/ml, about 120 pg/ml, about 130 pg/ml, about 140 pg/ml, about 150 pg/ml, about 160 pg/ml, about 170 pg/ml, about 180 pg/ml, or about 190 pg/ml of serum. In still other embodiments, the predetermined value of VEGF-A is about 200 pg/ml, about 210 pg/ml, about 220 pg/ml, about 230 pg/ml, about 240 pg/ml, about 250 pg/ml, about 260 pg/ml, about 270 pg/ml, about 280 pg/ml, or about 290 pg/ml of serum. In further embodiments, the predetermined value of VEGF-A is about 300 pg/ml, about 310 pg/ml, about 320 pg/ml, about 330 pg/ml, about 340 pg/ml, about 350 pg/ml, about 360 pg/ml, about 370 pg/ml, about 380 pg/ml, or about 390 pg/ml of serum. In some embodiments, the predetermined value of VEGF-A is about 400 pg/ml, about 410 pg/ml, about 420 pg/ml, about 430 pg/ml, about 440 pg/ml, about 450 pg/ml, about 460 pg/ml, about 470 pg/ml, about 480 pg/ml, or about 490 pg/ml of serum. In other embodiments, the predetermined value of VEGF-A is about 500 pg/ml of serum.

In some embodiments, the predetermined value of PF4 is about 20 pg/ml of serum. In other embodiments, the predetermined value of PF4 is about 30 pg/ml, about 40 pg/ml, about 50 pg/ml, about 60 pg/ml, about 70 pg/ml, about 80 pg/ml, or about 90 pg/ml. In yet other embodiments, the predetermined value of PF4 is about 100 pg/ml, about 110 pg/ml, about 120 pg/ml, about 130 pg/ml, about 140 pg/ml, about 150 pg/ml, about 160 pg/ml, about 170 pg/ml, about 180 pg/ml, or about 190 pg/ml of serum. In still other embodiments, the predetermined value of PF4 is about 200 pg/ml, about 210 pg/ml, about 220 pg/ml, about 230 pg/ml, about 240 pg/ml, about 250 pg/ml, about 260 pg/ml, about 270 pg/ml, about 280 pg/ml, or about 290 pg/ml of serum. In further embodiments, the predetermined value of PF4 is about 300 pg/ml, about 310 pg/ml, about 320 pg/ml, about 330 pg/ml, about 340 pg/ml, about 350 pg/ml, about 360 pg/ml, about 370 pg/ml, about 380 pg/ml, or about 390 pg/ml of serum. In some embodiments, the predetermined value of PF4 is about 400 pg/ml, about 410 pg/ml, about 420 pg/ml, about 430 pg/ml, about 440 pg/ml, about 450 pg/ml, about 460 pg/ml, about 470 pg/ml, about 480 pg/ml, or about 490 pg/ml of serum. In other embodiments, the predetermined value of PF4 is about 500 pg/ml of serum. In some embodiments, the predetermined value of PF4 is about 510 pg/ml, about 520 pg/ml, about 530 pg/ml, about 540 pg/ml, about 550 pg/ml, about 560 pg/ml, about 570 pg/ml, about 580 pg/ml, about 590 pg/ml, about 600 pg/ml, about 610 pg/ml, about 620 pg/ml, about 630 pg/ml, about 640 pg/ml, about 650 pg/ml, about 660 pg/ml, about 670 pg/ml, about 680 pg/ml, about 690 pg/ml, about 700 pg/ml about 710 pg/ml, about 720 pg/ml, about 730 pg/ml, about 740 pg/ml, about 750 pg/ml, about 760 pg/ml, about 770 pg/ml, about 780 pg/ml, or about 790 pg/ml, about 800 pg/ml 810 pg/ml, about 820 pg/ml, about 830 pg/ml, about 840 pg/ml, about 850 pg/ml, about 860 pg/ml, about 870 pg/ml, about 880 pg/ml, about 890 pg/ml, about 900 pg/ml, about 910 pg/ml, about 920 pg/ml, about 930 pg/ml, about 940 pg/ml, about 950 pg/ml, about 960 pg/ml, about 970 pg/ml, about 980 pg/ml, about 990 pg/ml, or about 1000 pg/ml of serum.

In some embodiments, the predetermined value of Angiostatin is about 5,000 pg/ml, about 5,500 pg/ml, about 6,000 pg/ml, about 6,500 pg/ml, about 7,000 pg/ml, about 7,500 pg/ml, about 8,000 pg/ml, about 8,500 pg/ml, about 9,000 pg/ml, about 9,500 pg/ml, about 10,000 pg/ml of serum. In other embodiments, the predetermined value of Angiostatin is about 15,000 pg/ml, about 15,500 pg/ml, about 16,000 pg/ml, about 16,500 pg/ml, about 17,000 pg/ml, about 17,500 pg/ml, about 18,000 pg/ml, about 18,500 pg/ml, about 19,000 pg/ml, about 19,500 pg/ml, about 20,000 pg/ml of serum.

In some embodiments, the predetermined value of Angiopoietin-1 is about 5,000 pg/ml of serum. In other embodiments, the predetermined value of Angiopoietin-1 is about 5,500 pg/ml, about 6,000 pg/ml, about 6,500 pg/ml, about 7,000 pg/ml, about 7,500 pg/ml, about 8,000 pg/ml, about 8,500 pg/ml, about 9,000 pg/ml, about 9,500 pg/ml, about 10,000 pg/ml of serum. In yet other embodiments, the predetermined value of Angiopoietin-1 is about 15,000, about 20,000 pg/ml, about 25,000 pg/ml, about 30,000 pg/ml, about 35,000 pg/ml, about 40,000 pg/ml, about 45,000 pg/ml, about 50,000 pg/ml, about 55,000 pg/ml, or about 60,000 pg/ml of serum.

In some embodiments, the predetermined value of Artemin is about 10 pg/ml of serum. In other embodiments, the predetermined value of Artemin is about 100 pg/ml, about 200 pg/ml, about 300 pg/ml, about 400 pg/ml, about 500 pg/ml, about 600 pg/ml, about 700 pg/ml, or about 800 pg/ml. In yet other embodiments, the predetermined value of Artemin is about 900 pg/ml, about 1000 pg/ml, about 1500 pg/ml, about 2000 pg/ml, about 2500 pg/ml, about 3000 pg/ml, about 3500 pg/ml, about 4000 pg/ml, about 4500 pg/ml, or about 5000 pg/ml of serum.

In some embodiments, the predetermined value of Angiopoietin-2 is about 1000 pg/ml of serum, about 1100 pg/ml, about 1200 pg/ml, about 1300 pg/ml, about 1400 pg/ml, about 1500 pg/ml, about 1600 pg/ml, about 1700 pg/ml, about 1800 pg/ml, about 1900 pg/ml, about 2000 pg/ml, about 2100 pg/ml, about 2200 pg/ml, about 2300 pg/ml, about 2400 pg/ml, about 2500 pg/ml, about 2600 pg/ml, about 2700 pg/ml, about 2800 pg/ml, about 2900 pg/ml, about 3000 pg/ml, about 3100 pg/ml, about 3200 pg/ml, about 3300 pg/ml, about 3400 pg/ml, about 3500 pg/ml, about 3600 pg/ml, about 3700 pg/ml, about 3800 pg/ml, about 3900 pg/ml, about 4000 pg/ml, about 4100 pg/ml, about 4200 pg/ml, about 4300 pg/ml, about 4400 pg/ml, about 4500 pg/ml, about 4600 pg/ml, about 4700 pg/ml, about 4800 pg/ml, about 4900 pg/ml, or about 5000 pg/ml of serum.

In some embodiments, the predetermined value of uPA is about 500 pg/ml, 600 pg/ml, 700 pg/ml, 800 pg/ml, 900 pg/ml, 1000 pg/ml, about 1100 pg/ml, about 1200 pg/ml, about 1300 pg/ml, about 1400 pg/ml, about 1500 pg/ml, about 1600 pg/ml, about 1700 pg/ml, about 1800 pg/ml, about 1900 pg/ml, about 2000 pg/ml, about 2100 pg/ml, about 2200 pg/ml, about 2300 pg/ml, about 2400 pg/ml, or about 2500 pg/ml of serum.

In some embodiments, the predetermined value of Vasohibin is about 20 pg/ml of serum to about 100 pg/ml of serum. In some embodiments, the predetermined value of Vasohibin is about 100 pg/ml of serum to about 1000 pg/ml of serum. In some embodiments, the predetermined value of Vasohibin is about 1000 pg/ml of serum to about 10,000 pg/ml of serum.

In some embodiments, the predetermined values of the biomarkers are determined using the R&D Systems Proteome Profiler™ Human Angiogenesis Array Kit (ARY007) (Minneapolis, Minn.) and reported in densitometric units. In some embodiments, the predetermined value of VEGF-C is a densitometric unit of about 270 (in some embodiments, 267 densitometric units). In some embodiments, the predetermined value of VEGF-A is a densitometric unit of about 2200 (in some embodiments, 2243 densitometric units). In some embodiments, the predetermined value of Angiostatin is a densitometric unit of about 23000 (in some embodiments, 22630 densitometric units). In some embodiments, the predetermined value of Angiopoietin-1 is a densitometric unit of about 470. The densitometric units can vary depending on the individual and analysis.

The predetermined value will depend, of course, upon the characteristics of the individual population in which the individual lies. In characterizing risk, numerous predetermined values can be established.

An individual or subject identified by the methods described herein refers to an organ transplant recipient (an individual or subject who has received an organ transplant, such as a heart, heart valve, kidney, liver, lung, bone, bone marrow, ligament, tendon, intestine, cornea, skin, or bladder transplant). An individual or subject includes a mammal, such as a human, non-human primate, cow, rabbit, horse, pig, sheep, goat, dog, cat, or rodent such a rat, mouse or a rabbit. In some of the preferred embodiments, the individual is a human.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Methods

### Patient Population

Adult heart transplant recipients followed in the ambulatory clinic at the Brigham and Women's Hospital were prospectively enrolled in this study. All patients were at least two years status-post orthotopic heart transplantation. Patients who had undergone heart re-transplantation or multiorgan transplantation were excluded. A total of 17 patients with cardiac allograph vasculopathy (CAV) (cases) and 16 age- and sex-matched control patients without CAV were enrolled over a 12-month period. Serum was obtained (e.g., collected, taken) from each patient and baseline demographic characteristics, indication for transplant, rejection history, current immunosuppression, and CMV serologic status were collected. Serum was frozen at minus 80 degrees Celsius on the same day the samples were drawn and stored until use. The protocol was approved by the Committee on Clinical Investigation at Children's Hospital Boston and written informed consent was obtained from all study subjects.

### Classification of CAV

The most recent coronary angiogram, hemodynamics, and echocardiogram were interpreted by clinicians caring for each patient at the Brigham and Women's Hospital and were used to identify patients for referral and enrollment. A single cardiologist investigator also reviewed cardiac testing results to ensure that subjects were properly classified. The angiograms were graded according to the published consensus guidelines from the International Society of Heart and Lung Transplantation (Mehra M. R. et al., J Heart Lung Transplant (2010) 29:717-727). The reviewer was blinded to the results of other clinical diagnostic testing performed on each patient.

### Profiling Levels of Angiogenesis Related Proteins

Concentrations of 55 angiogenesis related proteins were determined using a human angiogenesis protein array kit (ARY007, R&D Systems, Minneapolis, Minn.). Serum samples were assayed using standard techniques by the R&D Biomarker Testing Service blinded to patient data. Briefly, 0.5 mL of patient serum was added to a biotinylated detection antibody cocktail and was incubated with a nitrocellulose membrane spotted (in duplicate) with specific capture antibodies. The membranes were washed, incubated with streptavidin-horseradish peroxidase (HRP), and after an additional wash step were developed by chemiluminescence. Each blot was scanned using a transmission mode scanner and expression of each molecule was determined by densitometry using automated image analysis software. Densitometric values of duplicate samples were averaged and subtracted from the average densitometric value for each negative control to compensate for background. Values are reported in densitometric units (DU). Background levels of each molecule as well as undetectable levels were recorded as having a value of 0 DU. This assay allows for quantitative comparisons and ranking of patients based on levels of individual molecules.

### Statistical Methods

Patient characteristics between the cases and controls were compared using the Student t-test for normally distributed continuous variables, the Wilcoxon rank-sum test for continuous variables with a skewed distribution, and Fisher's exact test for proportions. Median levels of each serum biomarker were compared between cases and controls by the Mann-Whitney U test and displayed using box-and-whisker plots. Candidate biomarkers with P-values less than 0.05 in univariable analysis were entered into a backward stepwise multivariable logistic regression model to identify which candidate biomarkers were independently associated with CAV using the likelihood ratio test to assess significance. The equation is, as follows:

$$Probability of CAV = \frac{{e}^{{β}_{1} {X}_{1} + {β}_{2} {X}_{2} + {β}_{3} {X}_{3} + {β}_{0}}}{1 + {e}^{{β}_{1} {X}_{1} + {β}_{2} {X}_{2} + {β}_{3} {X}_{3} + {β}_{0}}}$$

where, (e) denotes the base of the natural logarithm, approximately 2.71, β (beta) values are the coefficients for the individual terms, and X values are the patient specific values for each molecule in densitometric units. X1=VEGF A, X2=VEGF C, and X3=PF4. β va Based on our multivariable logistic regression analysis:

$$Probability of CAV = \frac{{e}^{( 0.002 {X}_{1} + 0.051 {X}_{2} + 0.002 {X}_{3} 17.5 )}}{1 + {e}^{( 0.002 {X}_{1} + 0.051 {X}_{2} + 0.002 {X}_{3} 17.5 )}}$$

Receiver-operating characteristic (ROC) curve analysis was applied to determine diagnostic accuracy based on area under the curve (AUC) for
each significant multivariate predictor and composite of predictive biomarkers together. Statistical analysis was performed using SPSS software (SPSS Inc./IBM, Chicago, Ill.) and all reported P-values are two-tailed.

### Results

### Patient Characteristics

In the cohorts used, patients with CAV were 69% male and 88% Caucasian. The average age at transplant was 51±11 years and the average time since transplant was 11.3±3.9 years (Table 1). All patients with CAV had established disease with an average time from diagnosis of CAV to study enrollment of 5.7±3.5 years. All but one of the CAV patients were at least 1.5 years removed from the original diagnosis of CAV at the time of study enrollment. Forty-four percent of patients with CAV were transplanted for non-ischemic dilated cardiomyopathy. Coronary artery disease (25%) and congenital or valvular heart disease (19%) were the next most common indications. There were no significant differences between cases and controls with regards to sex, ethnicity, age at time of transplant, indication for transplant, time since transplant, and CAV status (Table 1). Donor age was significantly higher in patients with CAV (41±12 years vs. 31±12; P=0.02). Graft ischemic time and the number of episodes of acute rejection in the first post-transplant year were not significantly different between groups. The majority of patients in both groups were on triple drug immunosuppression with prednisone, cyclosporine, and azathioprine being the most frequently used combination. Among the patients with CAV, 9 (53%) had International Society for Heart & Lung Transplantation (ISHLT) Grade 1 disease, 3 (18%) had ISHLT Grade 2 disease, and 5 (29%) had ISHLT Grade 3 disease.

### CAV and Proteins Involved in Angiogenesis and Endothelial Proliferation

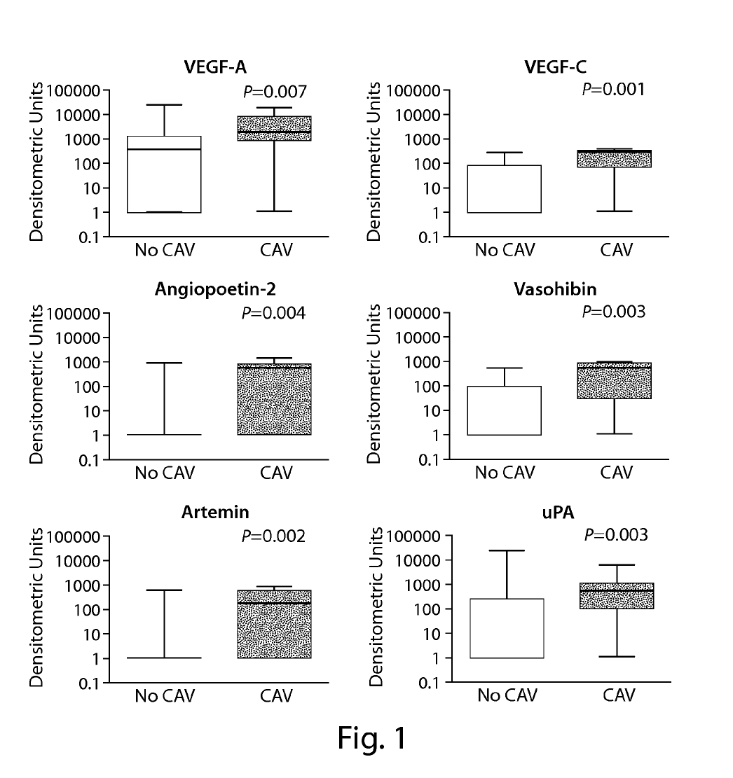

There were significant differences in the levels of 21 of the 55 angiogenesis related between patients with and without CAV (Table 2). Of these 21 proteins, vascular endothelial growth factor (VEGF)-C, Artemin, uPA, Vasohibin, Angiopoietin-2, and VEGF-A showed the greatest differences between cases and controls (FIG. 1). Data for other proteins are shown in Table 4.

### VEGF-C, VEGF-A, and PF4 as Sensitive and Specific Biomarkers for CAV

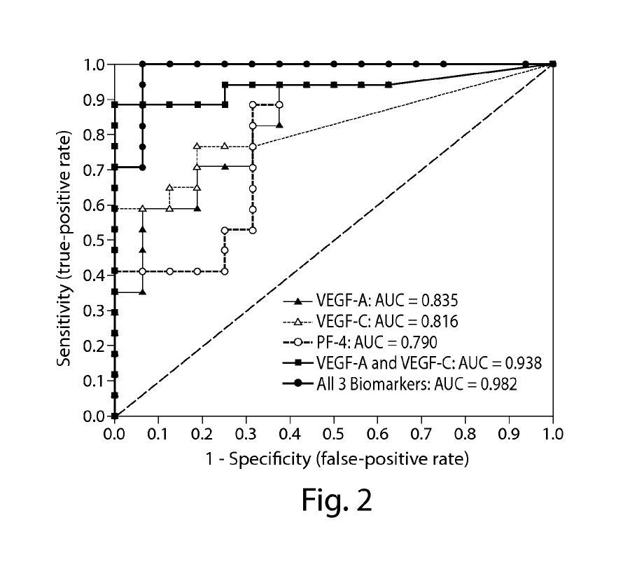

Multivariate logistic regression modeling identified VEGF-C, VEGF-A, and Platelet Factor 4 (PF4) as the strongest independent biomarkers associated with CAV. ROC analysis was then used to determine the diagnostic test characteristics of these biomarkers, both alone and in combination (FIG. 2 and Table 3). This analysis demonstrated that each individual biomarker had good test performance characteristics (range of area under the curve (AUC) values: 0.790-0.835; all P values<0.005); however, in combination the three biomarkers were excellent at identifying patients with CAV (AUC 0.982; 95% CI, 0.940-1.000; P<0.001).

### Discussion

In this study we demonstrated that soluble proteins involved in vascular remodeling are biomarkers of established CAV in patients with angiographically apparent disease. This study showed that VEGF-C, VEGF-A, and PF4 are highly sensitive and specific biomarkers for the identification of patients with CAV. These findings support the hypothesis that the endothelial cell response to alloimmune targeting/injury is a critical step in the progression of CAV, and further, provide evidence that serum levels of VEGF-C, VEGF-A, and PF4, form the basis for a clinical diagnostic test for CAV. We believe that serum levels of Artemin, uPA, Vasohibin, or Angiopoietin-2 are useful as diagnostic tests for CAV.

To date, no non-invasive blood test exists for use in CAV screening (Libby P, et al. J Clin Invest (2003) 107:1237-1239; Contreras A. G. et al. J Clin Invest (2007) 117:3645-3648). Chronic rejection with the development of CAV continues to be the most important cause of late allograft loss in heart transplant recipients (Stehlik J. et al. J Heart Lung Transplant (2011) 30:1078-1084) and its prevention and treatment is a priority for the development of novel therapeutics in the field. Unfortunately, a major impediment to progress relates to a lack of tools to predict disease initiation. Current state of the art continues to rely heavily on invasive testing, such as coronary angiography and IVUS (Schmauss D. et al. Circulation (2008) 117:2131-2141; Zimmer R. J. and Lee M. S. JACC Cardiovasc Interv (2010) 3:367-377; Cai Q. J. Cardiology in Review (2011) 19:30-35). While other less invasive imaging studies are available, including CT angiography, dobutamine stress echocardiography, and nuclear imaging, they have limitations due to their lack of sensitivity and/or their limited ability to detect early and small vessel disease (Schmauss D. et al (2008)). Thus, it is generally appreciated that sensitive and clinically useful biomarkers are needed to advance the ability to detect and treat this condition.

This study demonstrates that multiple proteins involved in angiogenesis are associated with CAV. We believe that these findings form the basis for the development of a quantitative blood based assay for the diagnosis of CAV. Consistent with other reports (Abramson L. P. et al. Transplantation (2002) 73:153-156; Bayliss J et al. Transplantation (2008) 86:991-997) high serum levels of VEGF-A were found in patients with (previously diagnosed with) angiographically apparent CAV. The data described herein shows that the combination of VEGF-A, VEGF-C and PF4 levels has high diagnostic test performance characteristics. In addition, related molecules including Angiopoetin-1, Artemin, Urokinase-type Plasminogen Activator, and Vasohibin are highly associated with CAV.

VEGF-A is an important pro-angiogenic molecule that is well established to promote the survival and proliferation of vascular endothelial cells. It also has potent pro-inflammatory effects which include its ability to act as a chemoattractant for monocytes and lymphocytes (Barleon B. et al. Blood (1996) 87:3336-3343; Reinders M. E. et al. J Clin Invest (2003) 112:1655-1665; Edelbauer M. et al. Blood (2010) 116:1980-1989) and its ability to elicit vascular permeability (Ferrara N. Eur Cytokine Netw (2009) 20:158-163). Consistent with our findings, over the past 10 years, VEGF-A has emerged as an important molecule in the rejection process, and its expression has been reported by our group as well as several others in association with both acute and chronic allograft rejection (Reinders et al. (2003); Reinders et al. J Am Soc Nephrol (2006) 17:931-942; Lemstrom et al. Circulation (2002) 105:2524-2530; Torry R. J. et al. Transplantation (1995) 60:1451-1457; Pilmore H. L. et al. Transplantation (1999) 67:929-933). Enhanced VEGF expression is confined to areas of the allograft myocardium in association with monocyte/macrophage infiltrates, and the expression of VEGF is associated with fibrin deposition (Tony R. J. et al (1995)). Increases in VEGF expression is observed within human cardiac allografts, and the expression of VEGF is spatially associated with infiltrates as well as graft vascular endothelial cells. Furthermore, high levels of VEGF-A expression correlate with the development of both acute and chronic allograft rejection, and persistent overexpression of intragraft VEGF-A identifies risk for the development of CAV (Reinders M. S. et al. Transplantation (2003) 76:224-230). Genotypes associated with high VEGF production confer increased risk for the development of chronic rejection (Girnita D. M. Transplantation (2008) 85:1632-1639; Girnita D. M. Transplantation (2011) 91:1326-1332; Shahbazi M. et al. J Am Soc Nephrol (2002) 13:260-264. Finally, in experimental animal models, VEGF-A is involved in the development of CAV. These models have shown that forced overexpression of VEGF within the myocardium of cardiac allografts results in monocyte recruitment, vascular disease and CAV development (Lemstrom K. B. et al. (2002). Taken together, the findings presented herein, as well as other data, provide compelling evidence that VEGF-A is expressed in cardiac allografts in association with rejection and that it may serve as a reliable biomarker of alloimmune-mediated graft injury as well as CAV disease activity.

An intriguing finding in this study is that VEGF-C is also associated with CAV. VEGF-C binds VEGF receptor (R)-2 and VEGFR-3 to mediate its biological effects, and it is well established to be a dominant factor stimulating lymphangiogenesis. Consistent with findings described herein, increased production of VEGF-C has been found in experimental models of chronic allograft rejection (Nykanen A. I. et al. Circulation (2010) 121:1413-1422) and lymphangiogenesis has been linked to chronic allograft rejection following both kidney and lung transplantation in humans (Kerjaschki D. et al. J Am Soc Nephrol (2004) 15:603-612; Dashkevich A. et al. Annals of Thoracic Surgery (2010) 90:406-412; Thaunat O. et al. Journal of Immunology (2010) 185:717-728). In addition, inhibition of VEGFR-3 leads to a decrease in lymphatic vessel activation, intragraft inflammation, and graft vasculopathy in a rat and mouse model of chronic cardiac rejection (Nykanen A. I. (2010)). The data presented herein provides the first clinical evidence that measurement of serum levels of VEGF-C may provide useful information following heart transplantation. Based on these data in both animals and humans, it may be that VEGF-C/VEGFR-3 interactions represent an important area for mechanistic investigation. Furthermore, these findings indicate that monitoring VEGF-A and VEGF-C together may serve as a highly specific and sensitive screen for CAV.

It is likely that a multiplex bead Luminex or ELISA-based assay will allow for more quantitative results and standardization of the data presented herein. Indeed, in a pilot analysis, a multiplex Luminex assay was performed to assess levels of VEGF-A in a subset of the cohort used herein. Using this technique, an increase in VEGF-A concentrations was found in patients with CAV compared to controls. A VEGF-A ELISA was also performed on approximately half of the serum samples, and again a close correlation with the protein profiler array was found. Finally, the study examined serum levels of these angiogenesis factors.

The current study revealed significant differences in multiple proteins involved in EC injury and repair responses. ROC analysis suggests that testing for VEGF-A and VEGF-C in combination is a highly sensitive method to screen for the presence of CAV. The use of additional biomarkers, such as PF4, enhances the diagnostic test performance characteristics in the small cohort used. We believe that monitoring blood concentrations of these proteins can be used to non-invasively diagnose CAV.

This discovery biomarker study has allowed for the identification of distinct patterns of soluble proteins associated with EC injury and repair, angiogenesis and endothelial proliferation in stable adult heart transplant recipients with angiographically apparent CAV.

### Example 2

A cross sectional cohort study was conducted, enrolling adult heart transplant recipients during routine outpatient follow up visits. Patients were determined to have cardiac allograft vasculopathy based on the presence of greater than 50% stenosis in at least one vessel on their most recent coronary angiogram. A total of 17 patients with cardiac allograft vasculopathy (cases) and 16 age and sex matched heart transplant recipients without cardiac allograft vasculopathy (controls) were enrolled. Table 5 lists patient characteristics of those enrolled in a cross sectional cohort study of heart transplant recipients (*Fisher Exact Test; ∥Student t-test; ΠWilcoxon Rank-Sum).

Serum and plasma were obtained (e.g., collected, taken) from each patient and baseline demographic characteristics, rejection history, current immunosuppression, indication for transplant, and CMV serologic status were collected. Serum and plasma were spun down, separated into aliquots, and frozen at minus 80 degrees Celsius on the same day the samples were drawn.

Concentrations of 55 angiogenesis related proteins were determined using the R&D Systems Proteome Profiler™ Human Angiogenesis Array Kit (ARY007) (Minneapolis, Minn.), incorporated herein by reference. The samples were run according to the protocol provided by the manufacturer. Depending on sample availability between 0.5 and 1 ml of serum were incubated with the nitrocellulose membrane antibody capture membrane. Each analyte was present in duplicate on the capture membrane. At the end of the assay the membrane was exposed to x-ray film, the film was electronically scanned using a transmission mode scanner and the pixel density was determined using image analysis software. Since each analyte was run in duplicate, the pixel density of the two spots for each analyte was averaged. The averaged value of the negative control spots were subtracted from the pixel density of each analyte to account for background. Negative values were recorded as non-detectable. Values were reported in densitometric units. No standard curve exists for this assay however it does allow comparison and ranking between patients/assays. Patient characteristics between the cases and controls were compared using the Student t-test for normally distributed continuous variables, the Wilcoxon Rank-Sum test for continuous variables with a skewed distribution, and the Fisher Exact Test for categorical variables.

In order to create an optimal prediction algorithm for diagnosis of cardiac allograft vasculopathy, a Classification and Regression Tree (CART) approach was undertaken using SPSS software. The presence of cardiac allograft vasculopathy was defined as the dependent variables and levels of all 55 proteins were entertained as independent predictor variables. The maximum tree depth was set at 5 with a minimum number of 2 cases in the parent node and 1 case in the child node.

CART analysis identified rules utilizing optimal cut-off levels of VEGF-C, VEGF-A, Angiopoietin-1, and Angiostatin that were 100% sensitive and 100% specific for the diagnosis of chronic rejection.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Each of the foregoing patents, patent applications and references is hereby incorporated by reference, particularly for the teaching referenced herein.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

TABLE 1
Patient, Donor, and Graft Characteristics

| | CAV | No CAV | |
|---|---|---|---|
| | (n = 17) | (n = 16) | P |
| Patient Characteristics | | | |
| Male Gender | 11 (69%) | 14 (82%) | 0.44 |
| Ethnicity | | | 1 |
| Caucasian | 14 (88%) | 15 (88%) | |
| African American | 0 (0%) | 1 (6%) | |
| Hispanic | 1 (6%) | 1 (6%) | |
| Asian/Pacific Islander | 1 (6%) | 0 (0%) | |
| Age at time of Transplant, y | 51.0 ± 10.8 | 48.2 ± 12.1 | 0.48 |
| Indication for Transplant | | | 0.86 |
| (n = 32) | | | |
| Coronary Artery Disease | 4 (25%) | 5 (31%) | |
| Dilated Cardiomyopathy: Ischemic | 1 (6%) | 1 (6%) | |
| Dilated Cardiomyopathy: Other | 7 (44%) | 4 (25%) | |
| Hypertrophic Cardiomyopathy | 1 (6%) | 2 (13%) | |
| Restrictive Cardiomyopathy | 0 (0%) | 1 (6%) | |
| Congenital or Valvular Heart | 3 (19%) | 3 (19%) | |
| Disease | | | |
| CAD Related Indication for | 5 (29%) | 7 (44%) | 0.48 |
| Transplant (n = 32) | | | |
| Recipient CMV Status IgG | 8 (67%) | 5 (63%) | 1 |
| Positive (n = 20) | | | |
| Donor and Graft Characteristics | | | |
| (n = 31) | | | |
| Male Gender | 7 (44%) | 9 (60%) | 0.48 |
| Donor Age | 41.4 ± 12.2 | 30.9 ± 11.6 | 0.02 |
| Donor CMV Status IgG Positive | 5 (33%) | 4 (25%) | 0.70 |
| Graft Ischemic Time (minutes) | 175 ± 54 | 152 ± 48 | 0.23 |
| Patient Characteristics at Time | | | |
| of Study Sample | | | |
| Time Since Transplant, y | 11.3 ± 3.9 | 12.4 ± 5.2 | 0.51 |
| Time from CAV Diagnosis to | 5.7 ± 3.5 | N/A | |
| Sample, y | | | |
| Number of Episodes of Acute | 1 [0, 1] | 1 [0, 5] | 0.19 |
| Rejection in 1^{st} year Post-Transplant | | | |
| (ISHLT Grade 3A or Greater) | | | |
| Immunosuppression at Time of | | | |
| Study | | | |
| Prednisone | 17 (100%) | 15 (94%) | 0.49 |
| Tacrolimus | 3 (18%) | 0 (0%) | 0.23 |
| Cyclosporine | 12 (71%) | 14 (88%) | 0.40 |
| Sirolimus | 5 (29%) | 1 (6%) | 0.18 |
| Mycophenolate mofetil | 5 (29%) | 6 (38%) | 0.72 |
| Azathioprine | 7 (41%) | 8 (50%) | 0.73 |

CAD, coronary artery disease; CMV, cytomegalovirus; ISHLT, International Society for Heart and Lung Transplantation; CAV, cardiac allograft vasculopathy

TABLE 2
Levels of angiogenesis-related proteins which showed statistically
significant differences in univariable analysis.

| | CAV | No CAV | |
|---|---|---|---|
| Protein | (n = 17) | (n = 16) | P |
| VEGF-C | 274 [63, 328] | 0 [0, 99] | 0.001 |
| Artemin | 157 [0, 579] | 0 [0, 0] | 0.002 |
| uPA | 590 [103, 1192] | 0 [0, 272] | 0.003 |
| Vasohibin | 500 [28, 851] | 0 [0, 96] | 0.003 |
| Angiopoietin-2 | 520 [0, 799] | 0 [0, 0] | 0.004 |
| VEGF-A | 1951 [806, 7902] | 372 [0, 1423] | 0.007 |
| Endothelin-1 | 434 [0, 908] | 0 [0, 116] | 0.01 |
| Thrombospondin-2 | 459 [10, 983] | 0 [0, 237] | 0.01 |
| Amphiregulin | 353 [0, 752] | 0 [0, 136] | 0.02 |
| TGFβ1 | 492 [67, 719] | 46 [0, 138] | 0.02 |
| CCL3 | 757 [37, 1393] | 18 [0, 470] | 0.02 |
| Persephin | 328 [0, 737] | 0 [0, 33] | 0.02 |
| PF4 | 1066 [484, 8840] | 84 [0, 2548] | 0.02 |
| SerpinB5 | 704 [43, 1358] | 0 [0, 421] | 0.02 |
| Thrombospondin-1 | 718 [0, 1200] | 0 [0, 170] | 0.02 |
| Epidermal Growth | 943 [0, 1540] | 0 [0, 566] | 0.03 |
| Factor | | | |
| FGF1 (Acidic) | 433 [0, 661] | 0 [0, 252] | 0.04 |
| FGF2 (Basic) | 290 [26, 531] | 0 [0, 117] | 0.04 |
| HBEGF | 830 [109, 1842] | 209 [0, 816] | 0.04 |
| TYMP | 683 [44, 1099] | 54 [0, 328] | 0.04 |
| SerpinF1 | 1115 [188, 1639] | 287 [0, 769] | 0.04 |

The densitometric value for each molecule is presented as the median [25^{th} percentile, 75^{th} percentile]. Comparisons were made using the Mann-Whitney U-test. CAV, cardiac allograft vasculopathy; FGF1, Fibroblast growth factor 1; FGF2, Fibroblast growth factor 2; HBEGF, Heparin-binding EGF-like growth factor; TGFβ1, transforming growth factor, beta 1; CCL3, Chemokine (C-C motif) ligand 3; TYMP, Thymidine phosphorylase; SerpinB5, Serpin peptidase inhibitor, clade B (ovalbumin), member 5; PF4, Platelet factor 4; SerpinF1, Serpin peptidase inhibitor, clade F (alpha-2 antiplasmin, pigment epithelium derived factor), member 1; uPA, Plasminogen activator, urokinase; VEGF-A, vascular endothelial growth factor A; VEGF-C, vascular endothelial growth factor C.

TABLE 3
Receiver-Operating Characteristic (ROC) Curve Analysis
for Biomarkers with the Strongest Independent Predictive
Value in the Multivariable Logistic Model.

| Biomarker | AUC | 95% CI | P |
|---|---|---|---|
| VEGF-A | 0.835 | 0.700–0.973 | <0.001 |
| VEGF-C | 0.816 | 0.665–0.967 | 0.002 |
| PF-4 | 0.790 | 0.632–0.949 | 0.004 |
| VEGF-A and VEGF-C | 0.938 | 0.840–0.999 | <0.001 |
| All 3 combined* | 0.982 | 0.942–1.000 | <0.001 |

AUC, area under the curve; CI, confidence interval; VEGF-A, vascular endothelial growth factor A; VEGF-C, vascular endothelial growth factor C; PF4, Platelet factor 4;
*Based on multivariable logistic regression analysis (combined AUC value is equivalent to the c-index).

TABLE 4
Additional angiogenesis-related proteins.

| | CAV | No CAV | |
|---|---|---|---|
| Protein | (n = 17) | (n = 16) | P |
| Activin A | 563 [0, 1178] | 0 [0, 215] | 0.06 |
| ADAMTS1 | 451 [0, 874] | 0 [0, 213] | 0.05 |
| Angiogenin | 41575 [37806, 44161] | 41223 [40333, 42578] | 0.83 |
| Angiopoietin 1 | 13308 [8097, 20076] | 10765 [5496, 18022] | 0.41 |
| Angiostatin | 1450 [932, 5445] | 668 [0, 5667] | 0.34 |
| CCL2 | 0 [0, 170] | 0 [0, 85] | 0.65 |
| CXCL16 | 12615 [9283, 24696] | 14012 [3186, 21806] | 0.64 |
| Dipeptidyl | 20669 [13146, 28129] | 16935 [7546, 23642] | 0.13 |
| Peptidase 4 | | | |
| EG-VEGF | 522 [0, 1130] | 0 [0, 232] | 0.06 |
| Endoglin | 13055 [3571, 25283] | 13572 [1689, 25973] | 0.94 |
| Endostatin | 14644 [4653, 25150] | 10354 [4040, 24368] | 0.8 |
| FGF 4 | 147 [24, 391] | 19 [0, 201] | 0.12 |
| FGF 7 | 0 [0, 123] | 0 [0, 57] | 0.62 |
| GDNF | 674 [3, 1228] | 0 [0, 605] | 0.07 |
| GM-CSF | 751 [0, 1323] | 0 [0, 529] | 0.1 |
| HGF | 704 [0, 1186] | 0 [0, 249] | 0.05 |
| IGFBP1 | 29193 [27318, 34246] | 31055 [23846, 33328] | 0.72 |
| IGFBP2 | 24073 [19290, 32642] | 28172 [21126, 31932] | 0.59 |
| IGFBP3 | 31383 [25466, 34882] | 30018 [21515, 33825] | 0.01 |
| IL-1β | 438 [0, 933] | 0 [0, 179] | 0.09 |
| IL-8 | 339 [0, 842] | 0 [0, 127] | 0.11 |
| Leptin | 24974 [771, 39857] | 28516 [22273, 41314] | 0.36 |
| MMP8 | 15896 [10244, 20804] | 11175 [4933, 18986] | 0.24 |
| MMP9 | 44121 [41343, 46811] | 41216 [39216, 45263] | 0.11 |
| NRG1-β1 | 725 [0, 1197] | 26 [0, 325] | 0.1 |

TABLE 5

| | CAV | No CAV | |
|---|---|---|---|
| | (n = 17) | (n = 16) | |
| | Mean ± SD | Mean ± SD | p-value |
| Sex | | | 0.44* |
| Male | 11 (69%) | 14 (82%) | |
| Female | 5 (31%) ∥ | 3 (18%) | |
| Age at Time of Transplant | 51.0 ± 10.8 | 48.2 ± 12.1 | 0.48¶ |
| (yrs) | | | |
| Time Since Transplant (yrs) | 11.3 ± 3.9 | 12.4 ± 5.2 | 0.51¶ |
| Time to Development of CAV | 5.5 ± 2.8 | N/A | |
| (yrs) | | | |
| Indication for Transplant | | | 0.86* |
| (n = 33) | | | |
| Coronary Artery Disease | 4 (25%) | 5 (31%) | |
| Dilated Cardiomyopathy: Ischemic | 1 (6%) | 1 (6%) | |
| Dilated Cardiomyopathy: Other | 7 (44%) | 4 (25%) | |
| Hypertrophic Cardiomyopathy | 1 (6%) | 2 (13%) | |
| Restrictive Cardiomyopathy | 0 (0%) | 1 (6%) | |
| Congenital or Valvar Heart | 3 (19%) | 3 (19%) | |
| Disease | | | |

What is claimed is:

1. A method, comprising:
(i) determining a level(s) of vascular endothelial growth factor-C(VEGF-C), vascular endothelial growth factor-A (VEGF-A), platelet factor-4 (PF4), Artemin, urokinase plasminogen activator (uPA), Vasohibin, or Angiopoietin-2, or any combination thereof in a sample from an organ transplant recipient individual;
(ii) comparing the level(s) of VEGF-C, VEGF-A, PF4, Artemin, uPA, Vasohibin, or Angiopoietin-2, or any combination thereof in the sample to a predetermined value(s) for VEGF-C, VEGF-A, PF4, Artemin, uPA, Vasohibin, or Angiopoietin-2, or any of the combinations thereof, respectively; and
(iii) identifying the individual as having or at increased risk of developing chronic organ transplant rejection if the level(s) of VEGF-C, VEGF-A, PF4, Artemin, uPA, Vasohibin, or Angiopoietin-2, or any of the combinations thereof in the sample is above the predetermined value(s) for VEGF-C, VEGF-A, PF4, Artemin, uPA, Vasohibin, or Angiopoietin-2, or any of the combinations thereof, respectively.

2. The method of claim 1, comprising assaying for the level(s) of VEGF-C, VEGF-A, PF4, Artemin, uPA, Vasohibin, or Angiopoietin-2, or any of the combinations thereof in the sample.

3. The method of claim 1, wherein the combination is VEGF-C and VEGF-A.

4. The method of claim 1, wherein the combination is VEGF-C, VEGF-A, and PF4.

5. The method of claim 1, wherein the sample is tissue, tissue aspirate, or bodily fluid.

6. The method of claim 5, wherein the sample is blood, plasma, or serum.

7. (canceled)

8. (canceled)

9. The method of claim 1, wherein the level(s) is a protein level or a nucleic acid level.

10. (canceled)

11. The method of claim 1, wherein the organ is heart, heart valve, kidney, liver, lung, bone, bone marrow, ligament, tendon, intestine, cornea, skin, or bladder.

12-27. (canceled)

28. A method, comprising:
(i) determining a level of vascular endothelial growth factor-C(VEGF-C) in a sample from an organ transplant recipient individual;
(ii) comparing the level of VEGF-C in the sample to a predetermined value for VEGF-C; and
(iii) identifying the individual as having or at increased risk of developing chronic organ transplant rejection if the level of VEGF-C in the sample is above the predetermined value for VEGF-C; or determining a level of a biomarker other than VEGF-C if the level of VEGF-C in the sample is at or below the predetermined for VEGF-C to identify if the individual has or is at increased risk of developing chronic organ transplant failure.

29. The method of claim 28, wherein the biomarker other than VEGF-C is VEGF-A.

30. The method of claim 28, comprising assaying for the level of VEGF-C and/or the level of VEGF-A in the sample.

31. The method of claim 29, further comprising
(i) comparing a level of VEGF-A in the sample to a predetermined value for VEGF-A;
(ii) comparing a level of Angiostatin in the sample to a predetermined value for Angiostatin if the level of VEGF-A in the sample is above the predetermined value for VEGF-A, or comparing a level of Angiopoietin-1 in the sample to a predetermined value for Angiopoietin-1 if the level of VEGF-A in the sample is at or below the predetermined value for VEGF-A; and
(iii) identifying the individual as having or at increased risk of developing chronic organ transplant rejection if the level of Angiostatin in the sample is at or below the predetermined value for Angiostatin or if the level of Angiopoietin-1 in the sample is at or below the predetermined value for Angiopoietin-1.

32. The method of claim 31, comprising determining the level(s) of VEGF-A, Angiostatin, or Angiopoietin-1, or any combination thereof in the sample from the individual.

33. The method of claim 32, comprising assaying for the level(s) of VEGF-A, Angiostatin, or Angiopoietin-1, or any combination thereof in the sample from the individual.

34. The method of claim 28, wherein the sample is tissue, tissue aspirate, or bodily fluid.

35-39. (canceled)

40. The method of claim 28, wherein the organ is heart, heart valve, kidney, liver, lung, bone, bone marrow, ligament, tendon, intestine, cornea, skin, or bladder.

41-66. (canceled)

67. A method, comprising:
(i) determining a level of vascular endothelial growth factor-A (VEGF-A) in a sample from an organ transplant recipient individual;
(ii) comparing the level of VEGF-A in the sample to a predetermined value for VEGF-A;
(iii) comparing a level of Angiostatin in the sample to a predetermined value for Angiostatin if the level of VEGF-A in the sample is above the predetermined value for VEGF-A, or comparing a level of Angiopoietin-1 in the sample to a predetermined value for Angiopoietin-1 if the level of VEGF-A in the sample is at or below the predetermined value for VEGF-A; and
(iv) identifying the individual as having or at increased risk of developing chronic organ transplant rejection if the level of Angiostatin in the sample is at or below the predetermined value for Angiostatin or if the level of Angiopoietin-1 in the sample is at or below the predetermined value for Angiopoietin-1.

68. The method of claim 67, comprising determining the level(s) of Angiostatin, Angiopoietin-1, or both in the sample from the individual.

69. The method of claim 67, comprising assaying for the level(s) of VEGF-A, Angiostatin, or Angiopoietin-1, or any combination thereof in the sample from the individual.

70. The method of claim 67, wherein the sample is tissue, tissue aspirate, or bodily fluid.

71-106. (canceled)
